(19) United States
(12) Patent Application Publication
(10) Pub. No.: US 2019/0070257 A1
(43) Pub. Date: Mar. 7, 2019

(54) Methods for Prophylactically Preventing, Slowing the Progression of, or Treating Alzheimer's Disease

(71) Applicant: HDL Therapeutics, Inc., Vero Beach, FL (US)

(72) Inventors: Hollis Bryan Brewer, Jr., Potomac, MD (US); Michael M. Matin, Short Hills, NJ (US)

(21) Appl. No.: 15/909,765

(22) Filed: Mar. 1, 2018

Related U.S. Application Data
(63) Continuation-in-part of application No. 15/876,808, filed on Jan. 22, 2018.
(60) Provisional application No. 62/537,581, filed on Jul. 27, 2017.
(60) Provisional application No. 62/516,100, filed on Jun. 6, 2017.
(60) Provisional application No. 62/465,262, filed on Mar. 1, 2017.
(60) Provisional application No. 62/516,100, filed on Jun. 6, 2017.
(60) Provisional application No. 62/465,262, filed on Mar. 1, 2017.
(60) Provisional application No. 62/449,416, filed on Jan. 23, 2017.

(51) Int. Cl. A61K 38/17; A61P 25/28; C07K 14/775; A61K 49/00

(52) U.S. Cl. CPC A61K 38/1709; A61K 49/0004; C07K 14/775; A61P 25/28

(57) ABSTRACT

The present specification is directed to systems, apparatus and methods for prophylactically preventing, or for treating the progression of, Alzheimer's disease. The progression of, stabilizing, or improving Symptoms related to Alzheimer's Disease (AD) are treated by monitoring a pathophysiological change indicative of AD in a patient, based on the monitoring, determining if amyloid plaque is present in a perivascular space of the patient, optionally determining an extent of amyloid plaque in the perivascular space, and based on the presence of amyloid plaque in the perivascular space of the patient, determining a treatment protocol for the patient. The treatment protocol includes administering to the patient a high density lipoprotein composition derived from mixing a blood fraction with a lipid removing agent.

### CROSS-REFERENCE

The present application relies on U.S. Provisional Patent Application No. 62/465,262, entitled “Method for Treating Familial Hypercholesterolemia” and filed on Mar. 1, 2017, for priority.

The present application is a continuation-in-part of U.S. patent application Ser. No. 15/876,808, entitled “Methods for Treating Cholesterol-Related Diseases”, and filed on Jan. 22, 2018, which, in turn, relies on U.S. Provisional Patent Application No. 62/449,416, entitled “Method for Treating Familial Hypercholesterolemia” and filed on Jan. 23, 2017, for priority.

The present application relies on U.S. Provisional Patent Application No. 62/516,100, entitled “Methods for Treating Cholesterol-Related Diseases” and filed on Jun. 6, 2017, for priority.

The present application relies on U.S. Provisional Patent Application No. 62/537,581, entitled “Methods for Treating Cholesterol-Related Diseases” and filed on Jul. 27, 2017, for priority.

The above-mentioned applications are all incorporated herein by reference in their entirety.

### FIELD

The method of the present specification provides for successively repeated treatment procedure for selective removal of lipid from HDL to create a modified HDL particle while leaving LDL particles substantially intact and the administration of the modified HDL particle to an individual having Alzheimer's disease in order to delay, halt and stabilize, reverse or improve the progression of the disease or pathophysiologic process that leads to the symptoms related to Alzheimer's disease.

### BACKGROUND

Historically, the use of clinical criteria that defined later stages of Alzheimer's disease (AD), such as after the onset of severe and marked dementia, determined the patients that were enrolled in clinical trials exhibited both the cognitive changes typical of clinically evident AD and the degree of functional impairment associated with marked dementia. As the scientific understanding of AD has evolved, efforts have been made to incorporate additional diagnostic information in order to enroll a greater class of patients in clinical trials. This diagnostic information includes, to varying degrees, the use of biomarkers reflecting underlying pathophysiological changes, which allows for the enrollment of patients in which there may be no apparent functional impairment or no detectable clinical abnormality. These patients are categorized as early onset AD patients. In using a broader range of diagnostic information to assess the degree and extent of AD, it is possible to intervene much earlier in the disease process given the onset of pathophysiological changes that can be measured and that precede clinically evident findings. There is thus a need to delay, halt, and preferably reverse the pathophysiological process that leads to the initial clinical deficits presented by AD.

AD is determined using results from several tests to arrive at a differential diagnosis. Thus, there is no definitive diagnosis for AD. Research has indicated that familial hypercholesterolemia is an early risk factor for AD. It is theorized that LDL receptors are involved in increasing the risk of AD. It has been observed that certain individuals are predisposed to AD, as demonstrated by family history or by genetic testing. Given that there is no established treatment for AD once lesions are formed, it would be desirable to provide a prophylactic way to treat AD or prevent the onset of AD altogether.

Existing apheresis and extracorporeal systems for treatment of plasma constituents and therefore lipid-related diseases suffer from a number of disadvantages that limit their ability to be used in clinical applications. A need exists for improved systems, apparatuses and methods capable of removing lipids from blood components in order to provide treatments and preventative measures for AD.

While the methods to selectively delipidate HDL particles overcomes several of the limitations stated above, what is also needed is a method to selectively remove lipid from HDL particles and thereby create modified HDL particles with increased capacity to accept cholesterol, without substantially affecting LDL particles, in chronic diseases. What is also needed is a method to successively monitor effectiveness of the modified HDL particles in accepting cholesterol in order to monitor the progress of a treatment using imaging techniques such as CT Angiography. Additionally, what is needed is a method to treat AD or prevent the onset of AD.

### SUMMARY

The following embodiments and aspects thereof are described and illustrated in conjunction with systems, tools and methods, which are meant to be exemplary and illustrative, not limiting in scope.

In some embodiments, the present specification discloses a method for delaying a progression of, halting and stabilizing, or reversing and improving symptoms related to Alzheimer's Disease (AD) in a patient, comprising: monitoring a pathophysiological change indicative of AD in a patient; based on said monitoring, determining if amyloid plaque is present in a perivascular space of the patient; determining an extent of amyloid plaque in said perivascular space; and, based on the presence of amyloid plaque in the perivascular space of the patient, determining a treatment protocol for the patient, wherein the treatment protocol comprises administering to the patient a high density lipoprotein composition derived from mixing a blood fraction with a lipid removing agent.

Optionally, diagnostic imaging is used to determine the presence and extent of amyloid plaque in the perivascular space of the patient.

Optionally, the high density lipoprotein composition is derived by obtaining the blood fraction from the patient, wherein the blood fraction has high-density lipoproteins; mixing the blood fraction with the lipid removing agent to yield modified high-density lipoproteins; separating the modified high-density lipoproteins; and delivering the modified high-density lipoproteins to the patient.

Optionally, the method further comprises connecting the patient to a device for withdrawing blood; withdrawing blood from the patient; and separating blood cells from the blood to yield the blood fraction containing high density lipoproteins and low density lipoproteins.

Optionally, the modified high density lipoproteins have an increased concentration of pre-beta high density lipoproteins relative to the high density lipoproteins from the blood fraction prior to mixing.

Optionally, the pathophysiological change is indicated by an accumulation of plaque in the perivascular space of the patient resulting in cerebral amyloid angiopathy.

Optionally, the high density lipoprotein composition derived from mixing the blood fraction with the lipid removing agent is delivered to the patient via infusion therapy in a dosage ranging from 1 mg/kg to 250 mg/kg.

Optionally, the high density lipoprotein composition derived from mixing the blood fraction of the patient with the lipid removing agent is delivered to the patient via infusion therapy at a rate of 999 mL/hour or another rate determined best for the patient.

Optionally, the method further comprises determining a severity of AD in the patient using at least one of global functioning, cognitive functioning, activities of daily living, or behavioral assessments.

Optionally, after administering to the patient the high density lipoprotein composition, the patient experiences a halt in further accumulation or a decrease in the accumulation of amyloid plaque in the perivascular space.

Optionally, after administering to the patient the high density lipoprotein composition, a rate of degeneration of the patient's physiological and/or cognitive parameters indicative of AD stabilizes and does not experience a further decrease.

Optionally, after administering to the patient the high density lipoprotein composition, a rate of degeneration of the patient's physiological and/or cognitive parameters indicative of AD, slows down relative to a rate of degeneration of the patient's physiological and/or cognitive parameters indicative of AD before administering to the patient the high density lipoprotein composition.

Optionally, after administering to the patient the high density lipoprotein composition, the patient's physiological and/or cognitive symptoms indicative of AD improve relative to the patient's physiological and/or cognitive symptoms indicative of AD before administering to the patient the high density lipoprotein composition.

Optionally, the high density lipoprotein composition is derived by obtaining the blood fraction from an individual other than the patient, wherein the blood fraction has high-density lipoproteins; mixing the blood fraction with the lipid removing agent to yield modified high-density lipoproteins; separating the modified high-density lipoproteins; and delivering the modified high-density lipoproteins to the patient.

In some embodiments, the present specification discloses a method for delaying the progression of, halting and stabilizing, or reversing and improving symptoms related to Alzheimer's Disease (AD) in a patient, comprising: monitoring a pathophysiological change indicative of AD, or a potential future onset of AD, in the patient; based on said monitoring, determining if amyloid plaque is present in a perivascular space of the patient; based on the determination of the presence of amyloid plaque in the perivascular space of the patient, determining a treatment protocol for the patient, wherein the treatment protocol comprises administering to the patient a high density lipoprotein composition derived from mixing a blood fraction, having unmodified high density lipoproteins, with a lipid removing agent to yield modified high density lipoproteins, wherein the modified high density lipoproteins have an increased concentration of pre-beta high density lipoprotein relative to the unmodified high density lipoproteins.

Optionally, the composition is derived by obtaining the blood fraction from the patient; mixing the blood fraction with the lipid removing agent to yield the modified high-density lipoproteins; separating the modified high-density lipoproteins; and delivering the modified high-density lipoproteins to the patient.

Optionally, the method further comprises connecting the patient to a device for withdrawing blood; withdrawing blood from the patient; and separating blood cells from the blood to yield the blood fraction containing low density lipoproteins and the high density lipoproteins.

Optionally, the composition is derived by obtaining the blood fraction from an individual other than the patient; mixing the blood fraction with the lipid removing agent to yield the modified high-density lipoproteins; separating the modified high-density lipoproteins; and delivering the modified high-density lipoproteins to the patient.

In some embodiments, the present specification discloses a method for improving an impairment of cognitive function indicative of Alzheimer's Disease (AD) in a patient, comprising: determining if amyloid plaque is present in a perivascular space of the patient; determining an extent or severity of cognitive impairment in the patient using at least one of a global, cognitive, functional or behavioral assessment test; and, based on the determination of the presence of amyloid plaque in the perivascular space of the patient and said extent or severity of cognitive impairment in the patient, determining a treatment protocol for the patient, wherein the treatment protocol comprises administering to the patient a high density lipoprotein composition derived from mixing a blood fraction of the patient with a lipid removing agent.

Optionally, the method further comprises determining an extent of amyloid plaque in the perivascular space and determining the treatment protocol based at least in part on the determined extent of amyloid plaque.

Optionally, the modified high density lipoproteins have an increased concentration of pre-beta high density lipoprotein relative to high density lipoproteins from the blood fraction prior to mixing.

Optionally, the composition is derived by: obtaining the blood fraction from the patient; mixing said blood fraction with the lipid removing agent to yield modified high-density lipoproteins; separating said modified high-density lipoproteins; and delivering said modified high-density lipoproteins to said patient.

Optionally, the AD is indicated by at least one of homozygous familial hypercholesterolemia, heterozygous familial hypercholesterolemia, ischemic stroke, coronary artery disease, acute coronary syndrome, or peripheral arterial disease.

Optionally, periodically monitoring changes comprises monitoring changes within a period of three to six months.

Optionally, the mixing the blood fraction with a lipid removing agent yields modified high density lipoprotein that has an increased concentration of pre-beta high density lipoprotein relative to total protein.

The aforementioned and other embodiments of the present specification shall be described in greater depth in the drawings and detailed description provided below.

### BRIEF DESCRIPTION OF THE DRAWINGS

These and other features and advantages of the present specification will be appreciated, as they become better understood by reference to the following detailed description when considered in connection with the accompanying drawings, wherein:

FIG. 1A is a flow chart delineating the steps of treating cardiovascular diseases using the treatment systems and methods in accordance with embodiments of the present specification;

FIG. 1B is another flow chart delineating the steps of treating cholesterol-related diseases, such as Atheroembolic Renal Disease (AERD), using the treatment systems and methods in accordance with embodiments of the present specification;

FIG. 1C is a table illustrating the types of treatments that may be provided for different compositions of degenerative material determined from an analysis, in accordance with some embodiments of the present specification;

FIG. 2 is a schematic representation of a plurality of components used in accordance with some embodiments of the present specification to achieve the processes disclosed herein;

FIG. 3 is a pictorial illustration of an exemplary embodiment of a configuration of a plurality of components used in accordance with some embodiments of the present specification to achieve the processes disclosed herein;

FIG. 4 is a longitudinal transverse cross-sectional view of a cerebral blood vessel illustrating removal of beta amyloid by transport along a cerebral lymphatic perivascular pathway, in accordance with an embodiment of the present specification;

FIG. 5 is a longitudinal transverse cross-sectional view of a cerebral blood vessel illustrating amyloid accumulation in a cerebral lymphatic perivascular pathway of an individual having a high level of the ε4 allele, in accordance with an embodiment of the present specification;

FIG. 6A is a longitudinal transverse cross-sectional view of a cerebral blood vessel of an AD patient being treated for cerebral amyloid angiopathy (CAA), in accordance with an embodiment of the present specification;

FIG. 6B illustrates a mechanism of removal of beta amyloid molecules by infusing pre-β HDL particles within the blood vessel of FIG. 6A, in accordance with an embodiment of the present specification;

FIG. 6C shows modified pre-β HDL particles flowing through the blood stream of the blood vessel of FIG. 6A, in accordance with an embodiment of the present specification; and

FIG. 7 is a flowchart describing plurality of exemplary steps of a therapeutic protocol for treating an AD patient, in accordance with an embodiment of the present specification.

### DETAILED DESCRIPTION

The present specification relates to methods and systems for treating cholesterol-related diseases. Embodiments of the present specification monitor changes in one or more atheroma areas and volumes in a patient, regularly over a period of time. Atheroma areas and volumes are monitored using known imaging techniques, for lipid-containing degenerative material in stenosis.

In accordance with embodiments of the present specification, based on the results of the monitoring, treatment is provided if accumulated lipid-containing degenerative material is identified to be present and above a threshold value. The treatment is repeated each time the atheroma areas and volumes are monitored, at pre-defined time intervals, and accumulated lipid-containing degenerative material is identified to be present and above the threshold.

Embodiments of the present specification treat the condition through systems, apparatuses and methods useful for removing lipid from α-High Density Lipoprotein (α-HDL) particles derived primarily from plasma of the patient thereby creating modified HDL particles with reduced lipid content, particularly reduced cholesterol content. Embodiments of the present specification create these modified HDL particles with reduced lipid content without substantially modifying LDL particles. Embodiments of the present specification modify original α-HDL particles to yield modified HDL particles that have an increased concentration of pre-β HDL relative to the original HDL.

Further, the newly formed derivatives of HDL particles (modified HDL) are administered to the patient to enhance cellular cholesterol efflux and treat cardiovascular diseases and/or other lipid-associated diseases, including Atheroembolic Renal Disease (AERD). The regular periodic monitoring and treatment process renders the methods and systems of the present specification more effective in treating cardiovascular diseases including Homozygous Familial Hypercholesterolemia (HoFH), Heterozygous Familial Hypercholesterolemia (HeFH), Ischemic stroke, Coronary Artery Disease (CAD), Acute Coronary Syndrome (ACS), peripheral arterial disease (PAD), Renal Arterial Stenosis (RAS), and for treating the progression of Alzheimer's Disease.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention. In the description and claims of the application, each of the words “comprise” “include” and “have”, and forms thereof, are not necessarily limited to members in a list with which the words may be associated.

It should be noted herein that any feature or component described in association with a specific embodiment may be used and implemented with any other embodiment unless clearly indicated otherwise.

The term “fluid” may be defined as fluids from animals or humans that contain lipids or lipid containing particles, fluids from culturing tissues and cells that contain lipids and fluids mixed with lipid-containing cells. For purposes of this invention, decreasing the amount of lipids in fluids includes decreasing lipids in plasma and particles contained in plasma, including but not limited to HDL particles. Fluids include, but are not limited to: biological fluids; such as blood, plasma, serum, lymphatic fluid, cerebrospinal fluid, peritoneal fluid, pleural fluid, pericardial fluid, various fluids of the reproductive system including, but not limited to, semen, ejaculatory fluids, follicular fluid and amniotic fluid; cell culture reagents such as normal sera, fetal calf serum or serum derived from any animal or human; and immunological reagents, such as various preparations of antibodies and cytokines from culturing tissues and cells, fluids mixed with lipid-containing cells, and fluids containing lipid-containing organisms, such as a saline solution containing lipid-containing organisms. A preferred fluid treated with the methods of the present invention is plasma.

The term “lipid” may be defined as any one or more of a group of fats or fat-like substances occurring in humans or animals. The fats or fat-like substances are characterized by their insolubility in water and solubility in organic solvents. The term “lipid” is known to those of ordinary skill in the art and includes, but is not limited to, complex lipid, simple lipid, triglycerides, fatty acids, glycerophospholipids (phospholipids), true fats such as esters of fatty acids, glycerol, cerebrosides, waxes, and sterols such as cholesterol and ergosterol.

The term “extraction solvent” or “lipid removing agent” may be defined as one or more solvents used for extracting lipids from a fluid or from particles within the fluid. This solvent enters the fluid and remains in the fluid until removed by other subsystems. Suitable extraction solvents include solvents that extract or dissolve lipid, including but not limited to phenols, hydrocarbons, amines, ethers, esters, alcohols, halohydrocarbons, halocarbons, and combinations thereof. Examples of suitable extraction solvents are ethers, esters, alcohols, halohydrocarbons, or halocarbons which include, but are not limited to di-isopropyl ether (DIPE), which is also referred to as isopropyl ether, diethyl ether (DEE), which is also referred to as ethyl ether, lower order alcohols such as butanol, especially n-butanol, ethyl acetate, dichloromethane, chloroform, isoflurane, sevoflurane (1,1,1,3,3,3-hexafluoro-2-(fluoromethoxy) propane-d3), perfluorocyclohexanes, trifluoroethane, cyclofluorohexanol, and combinations thereof.

The term “patient” refers to animals and humans, which may be either a fluid source to be treated with the methods of the present invention or a recipient of derivatives of HDL particles and or plasma with reduced lipid content.

The term “HDL particles” encompasses several types of particles defined based on a variety of methods such as those that measure charge, density, size and immuno-affinity, including but not limited to electrophoretic mobility, ultracentrifugation, immunoreactivity and other methods known to one of ordinary skill in the art. Such HDL particles include but are not limited to the following: α-HDL, pre-β HDL (including pre-β1 HDL, pre-β2 HDL and pre-β3HDL), HDL2 (including HDL2a and HDL2b), HDL3, VHDL, LpA-I, LpA-II, LpA-I/LpA-II (for a review see Barrans et al., Biochemica Biophysica Acta 1300; 73-85,1996). Accordingly, practice of the methods of the present invention creates modified HDL particles. These modified derivatives of HDL particles may be modified in numerous ways including but not limited to changes in one or more of the following metabolic and/or physico-chemical properties (for a review see Barrans et al., Biochemica Biophysica Acta 1300; 73-85,1996); molecular mass (kDa); charge; diameter; shape; density; hydration density; flotation characteristics; content of cholesterol; content of free cholesterol; content of esterified cholesterol; molar ratio of free cholesterol to phospholipids; immuno-affinity; content, activity or helicity of one or more of the following enzymes or proteins: ApoA-I, ApoA-II, ApoD, ApoE, ApoJ, ApoA-IV, cholesterol ester transfer protein (CETP), lecithin; cholesterol acyltransferase (LCAT); capacity and/or rate for cholesterol binding, capacity and/or rate for cholesterol transport.

The term “fractional flow reserve” or “FFR” is used to refer to a measurement of pressure differences across a coronary artery stenosis (a narrowing, usually due to atherosclerosis) to determine the likelihood that the stenosis impedes oxygen delivery to the heart muscle. Fractional flow reserve is defined as the pressure after (distal to) a stenosis relative to the pressure before the stenosis and is presented as an absolute number. An FFR value of 0.70 means that a given stenosis causes a 30% drop in blood pressure. Thus, FFR is used to express the maximal flow down a vessel in the presence of stenosis compared to the maximal flow in the hypothetical absence of stenosis. A decrease in blood flow, which is measured in terms of blood pressure using FFR, results in a decrease in oxygen delivery via blood (blood oxygen delivery).

The term “blockage due to lipid content” is measured in a percentage and is used to refer to the extent of physical blockage in an artery.

### Cardiovascular Diseases

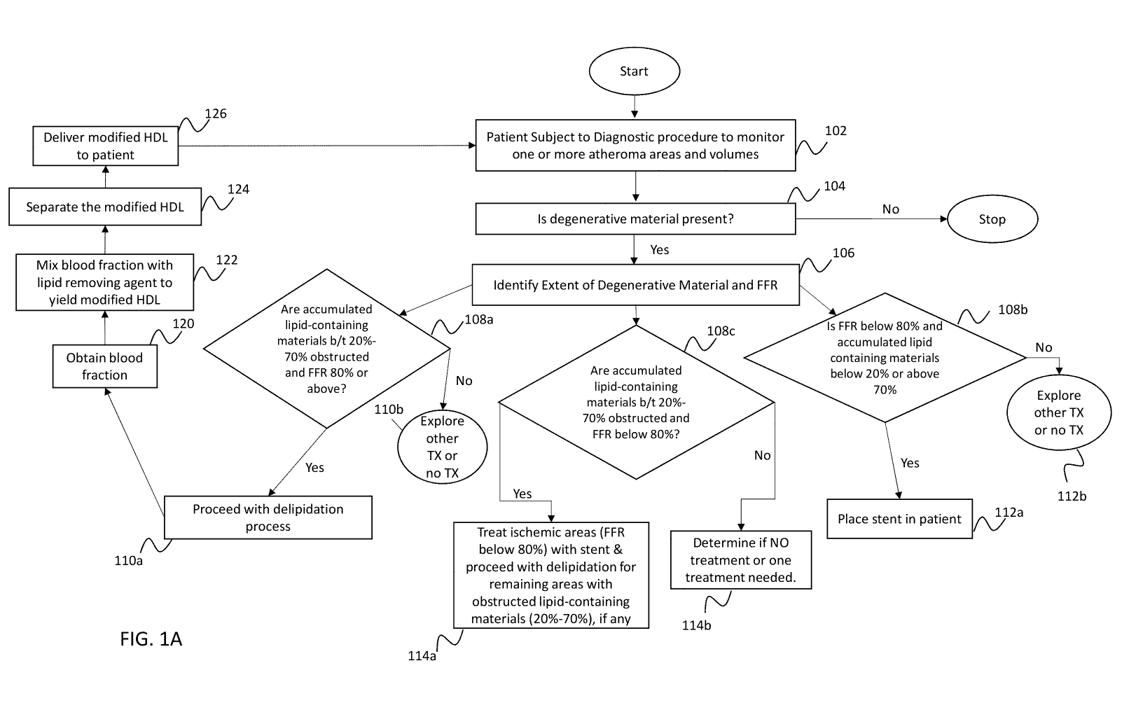

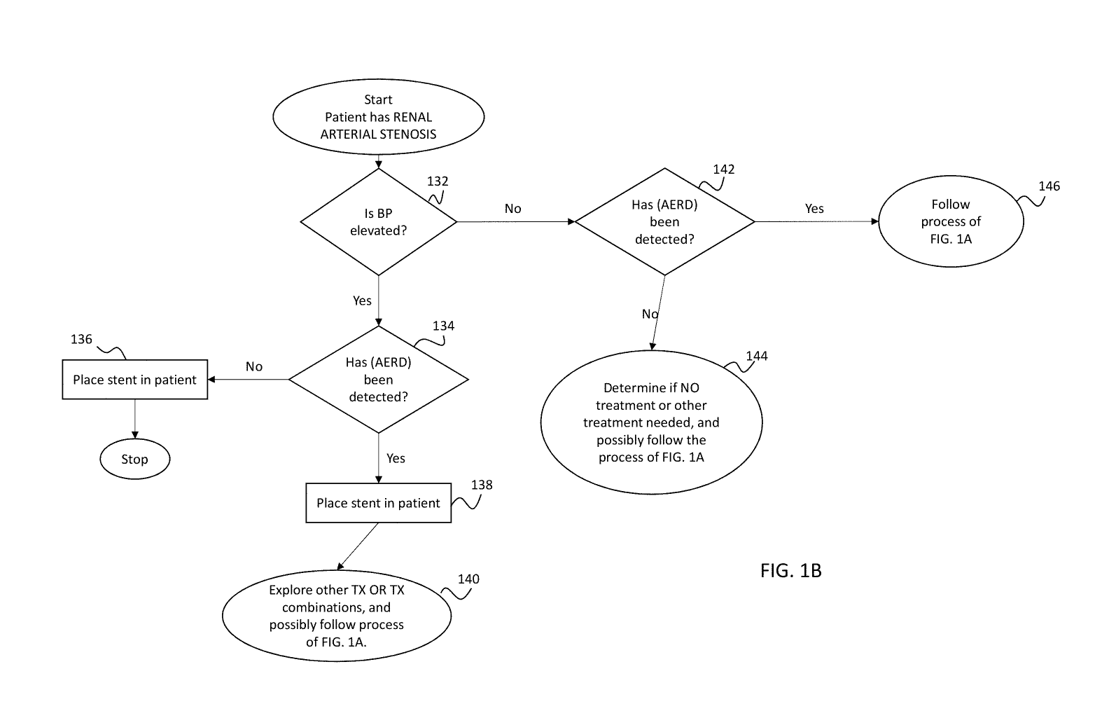

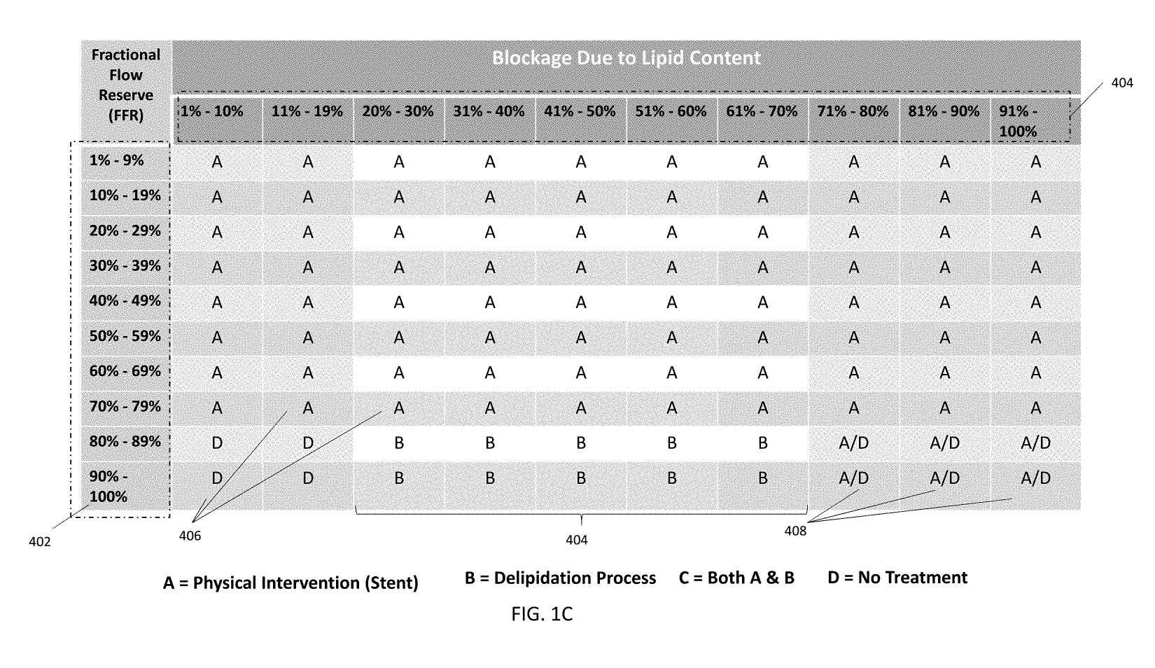

FIG. 1A is a flow chart illustrating an exemplary process of treating cardiovascular diseases, such as, but not limited to HoFH, HeFH, Ischemic stroke, CAD, ACS, peripheral arterial disease (PAD) and for treating the progression of Alzheimer's Disease in accordance with some embodiments of the present specification. At step 102, a subject or a patient who is diagnosed with a cardiovascular disease is monitored for one or more atheroma areas and/or volumes via a diagnostic procedure. In an embodiment, advanced medical imaging techniques, such as, but not limited to Computer Tomography (CT) angiogram and/or Intravascular Ultrasound (IVUS), may be used to detect areas within the inner layer of artery walls where lipid-containing degenerative material may have accumulated. Accumulated degenerative material may include fatty deposits which may include mostly macrophage cells, or debris, containing lipids, calcium and a variable amount of fibrous connective tissue. Analysis from the imaging techniques may also be used to identify and therefore monitor volumes of lipid-containing degenerative material accumulated within the inner layer of artery walls. Lipid-containing degenerative material and non-lipid-containing degenerative material may swell in the artery wall, thereby intruding into the channel of the artery and narrowing it, resulting in restriction of blood flow.

Based on analysis from the diagnostic technique, in step 104, the presence and type of degenerative material is confirmed. In addition, the extent or percentage blockage caused by degenerative material (lipid-containing or non-lipid-containing) is determined by a physician using diagnostic imaging techniques. If no degenerative material is detected at step 104, or if the level of degenerative material falls outside a pre-determined range of values, the process is stopped. In an embodiment, the physician identifies one or more arteries with stenosis that have a blockage of 20%-70% due to accumulated lipids, in order to implement treatment methods in accordance with the present specification. In step 106, a Fractional Flow Reserve (FFR) measurement is used to determine the extent of oxygen delivery in the presence of stenosis. In an embodiment, FFR is used to measure pressure differences across a coronary artery stenosis to determine the likelihood that the stenosis impedes blood oxygen delivery to the heart muscle (ischemia).

Different types of treatments may be provided depending on the diagnostic results and threshold values. At this stage, the physician may determine that either the treatment in accordance with embodiments of the present specification is not required as the disease has subsided, is not present, is not sufficient, or has been treated, or an alternative form of treatment (such as a physical stent) is required.

FIG. 1C is a table illustrating the types of treatments that may be provided for different compositions of degenerative material determined from the diagnosis for cardiovascular diseases, as described in the flow chart of FIG. 1A, in accordance with some embodiments of the present specification. The table compares different types of treatments that may be administered for combinations of various ranges of a Fractional Flow Reserve (FFR) 402, which is indicative of a rate of flow of blood after a blockage (which, in turn, is indicative of blood oxygen delivery), provided in terms of percentage (or fraction) of Fractional Flow Reserve, and various ranges of physical blockage due to lipid content 404, provided in terms of percentage of blockage due to lipid content. Referring to the table, each cell, such as cells 406, corresponds to a combination of a range 402 (indicative of FFR) and a range 404 (indicative of the percentage or extent of blockage due to lipid content), which further indicates at least one method of treatment that may be suitable for that combination.

In embodiments, the different types of treatments are coded as A, B, C, and D. Treatment type ‘A’ corresponds to an invasive treatment process where a stent is embedded through physical intervention. Treatment type ‘B’ corresponds to implementing the treatment methods of selectively modifying HDL particles, in accordance with the embodiments of the present specification. In an embodiment, it is preferable to selectively modify HDL particles (and perform the HDL infusions) where the Fractional Flow Reserve (FFR) ranges from 80-100% and the accumulated lipid obstruction ranges from 20-70%, as noted by sections 404. It should be noted herein that in embodiments, a FFR measurement of 1-79% represents an ischemic condition, wherein a FFR measurement of 80-100% represents a non-ischemic condition. In most cases, treatment types ‘A’ and/or ‘B’ may be able to address the condition. Treatment type ‘D’ corresponds to cases where neither of the stated treatment types (A and/or B) is required. In some cells, such as cells 408, two treatment options may be indicated and the physician would decide upon the appropriate course of treatment.

Treatment type ‘C’ corresponds to cases where a combination of both a stent as well as selective modification of HDL particles is administered (as described in greater detail below with respect to 114a in FIG. 1A). Atherosclerosis is a systemic disease and patients may have multiple lesions throughout their vasculature. Therefore, it should be noted herein that the treatment methods of the present specification are not implemented based on an overall patient health-based treatment strategy, but rather a “lesion/plaque/area/region”-based treatment strategy. Thus, in a few cases, a physician may decide to combine the treatments and administer treatment type ‘C’. If, in a particular patient, one or more areas or lesions have a FFR of 79% or less (ranging from 1% to 79%), then those areas would have a stent implanted. If the same patient presents with additional, remaining lesions that exhibit lipid-based blockage in the range of 20-70% and also an FFR of 80-100%, then the patient would undergo a subsequent delipidation. Therefore, both interventions may be used for patients having multiple lesions with different levels of disease at each lesion.

Referring again to FIG. 1A, at step 108a, a physician determines whether the amount of accumulated lipid-containing degenerative material, covering a lesion/plaque/area/region, falls above a predetermined threshold value or within a range of values, as measured in terms of a percentage of blockage due to lipid content. If arteries with atheroma lesion(s) having an amount or volume of lipid-containing material above the threshold percentage value or that fall within a range of values are not identified, an alternative treatment process (which may include no treatment or physical intervention) is determined by the physician, in step 110b. If arteries with lipid-containing atheroma lesion/plaque/area/region(s) having an amount or volume of lipid blockage above the predetermined threshold percentage or within a predetermined range of percentages are identified, the patient is then subjected to the delipidation process, in step 110a. The delipidation process of the present specification is described in greater detail below.

At step 108b, a physician determines whether, based on the FFR measurement, blood oxygen delivery is impeded below a threshold value or within a range of values (which is expressed as the maximal flow of blood down a vessel in the presence of stenosis compared to the maximal flow in the hypothetical absence of stenosis). If blood oxygen delivery is impeded below a threshold value or within a predetermined range of values, then in step 112a, a physician treats with physical intervention, such as a stent. In step 112b, if it is determined that blood oxygen delivery is not impeded below a threshold value or does not fall within a predetermined range of values, the physician explores an alternate treatment option (which may include no treatment or the delipidation process of the present specification). In an embodiment, the threshold value is 80%. In an embodiment, the range of values is 1%-79%.

At step 108c, a physician determines whether both the accumulated lipid-containing degenerative material, covering a lesion/plaque/area/region(s) is of an amount or volume falling within a predetermined range of percentages and blood oxygen delivery is impeded as determined by a predetermine range of percentages. If both conditions are met, in step 114a, the physician treats those areas identified as ischemic areas (FFR measurement in a range of 1% to 79%, and preferably below 80%) with a stent implant procedure and subsequently, the remaining areas with the delipidation process of the present specification. In step 114b, if both threshold conditions are not met, then the physician determines if either one of the conditions or neither condition is met and determines an appropriate course of treatment as outlined above.

In an example case, where the analysis from the imaging determines a FFR in the range of 1%-79%, and blockage due to lipids anywhere from 1 to 100%, a physician may decide to physically intervene to improve the blood flow as measured by FFR, and thus, blood oxygen delivery. In an embodiment, the physical intervention is performed by surgically embedding a stent in order to increase the rate of blood flow in the identified atheroma area.

In another example, where the analysis from the imaging determines a FFR in the range of 80%-100%, and blockage due to lipids to be in the range of 20%-70%, the physician may opt for treatment methods that remove or reduce the lipids. In this example, embodiments of the present specification that enable selective modification of HDL particles are utilized.

In yet another example, where the FFR is determined to be in a range of 1% to 79%, and preferably less than 80%, and blockage due to lipids is in the range of 20%-70%, the physician may opt to proceed with the surgical process of embedding a stent. It should be appreciated that when a percentage blockage is stated, such as 20%-70%, it means that a cross-sectional area of a vessel is blocked with lipid containing material and that such blockage occupies a range of 20% to 70% of the cross-sectional area of the vessel.

If arteries with lipid-containing atheroma lesion/plaque/area/region(s) having an amount or volume of lipid blockage within a predetermined range of percentages are identified in step 110a, the patient is then subjected to the delipidation process. In this case, at step 120, a blood fraction of the patient is obtained. The process of blood fractionation is typically done by filtration, centrifuging the blood, aspiration, or any other method known to persons skilled in the art. Blood fractionation separates the plasma from the blood. In one embodiment, blood is withdrawn from a patient in a volume sufficient to produce about 12 ml/kg of plasma based on body weight. The blood is separated into plasma and red blood cells using methods commonly known to one of skill in the art, such as plasmapheresis. Then the red blood cells are stored in an appropriate storage solution or returned to the patient during plasmapheresis. The red blood cells are preferably returned to the patient during plasmapheresis. Physiological saline is also optionally administered to the patient to replenish volume.

Blood fractionation is known to persons of ordinary skill in the art, and is performed remotely from the method described in context of FIG. 1A. During the fractionation, the blood can optionally be combined with an anticoagulant, such as sodium citrate, and centrifuged at forces approximately equal to 2,000 times gravity. The red blood cells are then aspirated from the plasma. Subsequent to fractionation, the cells are returned to the patient. In some alternate embodiments, Low Density Lipoprotein (LDL) is also separated from the plasma. Separated LDL is usually discarded. In alternative embodiments, LDL is retained in the plasma. In accordance with embodiments of the present specification, blood fraction obtained at 120 includes plasma with High Density Lipoprotein (HDL), and may or may not include other protein particles. In embodiments, autologous plasma collected from the patient is subsequently treated via an approved plasmapheresis device. The plasma may be transported using a continuous or batch process.

At step 122, the blood fraction obtained at 120 is mixed with one or more solvents, such as lipid removing agents. In an embodiment, the solvents used include either or both of organic solvents sevoflurane and n-butanol. In embodiments, the plasma and solvent are introduced into at least one apparatus for mixing, agitating, or otherwise contacting the plasma with the solvent. In embodiments, the solvent system is optimally designed such that only the HDL particles are treated to reduce their lipid levels and LDL levels are not affected. The solvent system includes factoring in variables such as solvent employed, mixing method, time, and temperature. Solvent type, ratios and concentrations may vary in this step. Acceptable ratios of solvent to plasma include any combination of solvent and plasma. In some embodiments, ratios used are 2 parts plasma to 1 part solvent, 1 part plasma to 1 part solvent, or 1 part plasma to 2 parts solvent. In an embodiment, when using a solvent comprising 95 parts sevoflurane to 5 parts n-butanol, a ratio of two parts solvent per one part plasma is used. Additionally, in an embodiment employing a solvent containing n-butanol, the present specification uses a ratio of solvent to plasma that yields at least 3% n-butanol in the final solvent/plasma mixture. In an embodiment, a final concentration of n-butanol in the final solvent/plasma mixture is 3.33%. The plasma and solvent are introduced into at least one apparatus for mixing, agitating, or otherwise contacting the plasma with the solvent. The plasma may be transported using a continuous or batch process. Further, various sensing means may be included to monitor pressures, temperatures, flow rates, solvent levels, and the like. The solvents dissolve lipids from the plasma. In embodiments of the present specification, the solvents dissolve lipids to yield treated plasma that contains modified HDL particles with reduced lipid content. The process is designed such that HDL particles are treated to reduce their lipid levels and yield modified HDL particles without destruction of plasma proteins or substantially affecting LDL particles.

Energy is introduced into the system in the form of varied mixing methods, time, and speed. At 124, bulk solvents are removed from the modified HDL particles via centrifugation. In embodiments, any remaining soluble solvent is removed via charcoal adsorption, evaporation, or Hollow Fiber Contractors (HFC) pervaporation. The mixture is optionally tested for residual solvent via use of chromatography (GC), or similar means. The test for residual solvent may optionally be eliminated based on statistical validation.

At 126, the treated plasma containing modified HDL particles with reduced lipid content, which was separated from the solvents at 124, is treated appropriately and subsequently returned to the patient. The modified HDL particles are HDL particles with an increased concentration of pre-beta HDL. Concentration of pre-beta HDL is greater in the modified HDL, relative to the original HDL that was present in the plasma before treating it with the solvent. The resulting treated plasma containing the HDL particles with reduced lipid and increased pre-beta concentration is optionally combined with the patient's red blood cells, if the red cells were not already returned during plasmapheresis, and administered to the patient. One route of administration is through the vascular system, preferably intravenously.

In embodiments, the patient is monitored again for changes in the previously monitored atheroma areas and volumes, specifically for lipid-containing degenerative material. Therefore the process is repeated from step 102, as described above. In embodiments, the patient is monitored repeatedly within a period of three to six months. The treatment cycle is also repeated at this frequency until the monitoring suggests substantially or completely enhanced cholesterol efflux. In an embodiment, when the atheroma area and volume are monitored to be below threshold, the patient may be considered to have been treated and may not require further repetition of the treatment cycle. In some embodiments, frequency of treatment may vary depending on the volume to be treated and the severity of the condition of the patient.

### Atheroembolic Renal Disease

Renal Arterial Stenosis (RAS) is a systemic disease and patients may have multiple lesions throughout their vasculature. Sometimes, the plaque within the arteries may break away and damage kidneys, resulting in Atheroembolic Renal Disease (AERD). Therefore, it should be noted herein that the treatment methods of the present specification are not implemented based on an overall patient health-based treatment strategy, but rather a “lesion/plaque/area/region”-based treatment strategy.

FIG. 1B is a flow chart illustrating another exemplary process of treating cholesterol-related diseases, such as, but not limited to Atheroembolic Renal Disease (AERD), in accordance with some embodiments of the present specification. In all cases, a patient first presents with renal arterial stenosis-a blockage in an artery that supplies blood to the kidney. At step 132, it is determined whether a patient has elevated Blood Pressure (BP). Recent onset of hypertension may be a clinical manifestation of the presence of plaque. If it is determined that the patient has High BP (HBP), the physician, may look for atheroembolic renal disease (AERD) at step 134. While AERD may not cause any symptoms, some of the following symptoms may appear slowly and worsen over time: blood in the urine, fever, muscle aches, headache, weight loss, foot pain or blue toes, nausea, among other symptoms. If AERD is not identified, then at 136, a stent is placed in the patient to reverse any blockage that may be resulting in HBP.

If, at 134, AERD is identified in addition to elevated BP, then the physician may place a stent at step 138 in order to reverse blockage and elevations in BP. Additionally, at step 140, the physician may determine whether the procedure of placing a stent has worked to address both elevated BP levels and AERD. If not, an additional stent may be placed, or the delipidation process, in accordance with embodiments of the present specification and described with respect to FIG. 1A, may be used. The treatment decision may be based on “lesion/plaque/area/region” determination.

At step 132, if it is determined that the patient has normal levels of BP, the physician may still check for symptoms or signs of AERD at step 142. The check may be conducted on the basis of symptoms such as, but not limited to, blindness, blood in the urine, fever, muscle aches, headache, weight loss, foot pain or blue toes, nausea, among other symptoms. If, at 142, AERD is not detected, then, at step 144, the physician may determine an appropriate course of treatment, based on the symptoms and any other diagnosis. If there is renal stenosis (the presence of cholesterol-containing plaque) absent both elevated HBP and AERD, then the physician may opt to follow the procedure outlined above in context of FIG. 1A for cardiovascular diseases, which can result in either one or both of a stent and/or the delipidation process of the present specification.

If the patient is diagnosed with AERD but has normal BP levels, then the physician may proceed to step 146, and the subject or the patient is monitored for one or more atheroma areas and/or volumes via a diagnostic procedure to determine the cause of renal dysfunction, and the extent of renal arterial stenosis. In an embodiment, advanced medical imaging techniques, such as, but not limited to Computer Tomography (CT) angiogram and/or Intravascular Ultrasound (IVUS) and/or Near IR spectroscopy, may be used to detect areas within the inner layer of artery walls where lipid-containing degenerative material may have accumulated. Accumulated degenerative material may include fatty deposits which may include mostly macrophage cells, or debris, containing lipids, calcium and a variable amount of fibrous connective tissue. Analysis from the imaging techniques may also be used to identify and therefore monitor volumes of lipid-containing degenerative material accumulated within the inner layer of artery walls. Lipid-containing degenerative material and non-lipid-containing degenerative material may swell in the artery wall, thereby intruding into the channel of the artery and narrowing it, resulting in restricting of blood flow and causing renal abnormalities.

Based on analysis from the diagnostic technique, the presence and type of degenerative material is confirmed, the extent or percentage of degenerative material (lipid-containing or non-lipid-containing) is determined, and the extent of blood oxygen delivery based on Fractional Flow Reserve (FFR) is identified. The process is stopped if no degenerative material is detected, or if the level of degenerative material is below a predetermined threshold or falls outside of a predetermined range of values. In an embodiment, the physician identifies one or more renal arteries with stenosis that have a blockage of 20%-70% due to accumulated lipids, in order to implement treatment methods in accordance with the present specification. In an embodiment, FFR is used to measure pressure differences across arterial stenosis to determine the likelihood that the stenosis impedes blood flow, and thus, oxygen delivery to the kidney (ischemia).

Different types of treatments may be provided depending on the diagnostic results and threshold values. At this stage, the physician may determine that either the treatment in accordance with embodiments of the present specification is not required as the disease has subsided, is not present, is not sufficient, or has been treated; or an alternative form of treatment is required.

Referring back to FIG. 1C, the table compares different types of treatments that may be administered for combinations of various ranges of a Fractional Flow Reserve (FFR) 402, which is indicative of a change in rate of flow of blood associated with a blockage (and thus blood oxygen delivery), provided in terms of percentage of FFR, and various ranges of blockage due to lipid content 404, provided in terms of percentage of blockage due to lipid content. Referring to the table, each cell, such as cells 406, correspond to a combination of a range 402 (indicative of FFR) and a range 404 (indicative of the percentage or extent of blockage due to lipid content), which further indicates at least one method of treatment that may be suitable for that combination.

In embodiments, the different types of treatments are coded as A, B, C, and D. Treatment type ‘A’ corresponds to an invasive treatment process where a stent is embedded through physical intervention. Treatment type ‘B’ corresponds to implementing the treatment methods of selectively modifying HDL particles, in accordance with the embodiments of the present specification. In an embodiment, it is preferable to selectively modify HDL particles (and perform the HDL infusions) where the Fractional Flow Reserve (FFR) ranges from 80-100% and the accumulated lipid obstruction ranges from 20-70%, as noted by sections 404. It should be noted herein that in embodiments, a FFR of 1-79% represents an ischemic condition, wherein 80-100% FFR represents a non-ischemic condition. Treatment type ‘C’ corresponds to cases where a combination of both a stent as well as selective modification of HDL particles is administered. In most cases, treatment types ‘A’ and/or ‘B’ may be able to address the condition. Treatment type ‘D’ corresponds to cases where neither of the stated treatment types (A, B, or C) is required. In some cells, such as cells 408, two treatment options may be indicated and the physician would decide upon the appropriate course of treatment.

Renal Arterial Stenosis (RAS) is a systemic disease and patients may have multiple lesions throughout their vasculature. It should be noted herein that the treatment methods of the present specification are not implemented based on an overall patient health-based treatment strategy, but rather a “lesion/plaque/area/region”-based treatment strategy. Thus, in a few cases, a physician may decide to combine the treatments and administer treatment type ‘C’. If, in a particular patient, one or more areas or lesions have a FFR percentage measured at 79% or less, then those areas would have a stent implanted. If the same patient presents with additional, remaining lesions that exhibit lipid-based blockage in the range of 20-70% and also an FFR of 80-100%, then the patient would undergo a subsequent delipidation. Therefore, both interventions may be used for patients having multiple lesions with different levels of disease at each lesion.

The physician determines whether the amount of accumulated lipid-containing degenerative material, covering a lesion/plaque/area/region, falls above or below a predetermined threshold percentage or within a predetermined range of percentages, as measured in terms of a percentage of blockage due to lipid content. If arteries with lipid-containing atheroma lesion(s) having an amount or volume above or below a threshold percentage or falling within a predetermined range of percentages are not identified, an alternative treatment process (which may include no treatment or physical intervention) is determined by the physician. If arteries with lipid-containing atheroma lesion/plaque/area/region(s) having an amount or volume of lipid blockage falling within a predetermined range of percentages are identified, the patient is then subjected to the delipidation process. The delipidation process of the present specification is described in greater detail with respect to FIG. 1A.

The physician also determines whether, based on the FFR measurement, blood oxygen delivery is impeded below a threshold value or falls within a range of values (which is expressed as the maximal flow of blood down a vessel in the presence of stenosis compared to the maximal flow in the hypothetical absence of stenosis). If blood oxygen delivery is impeded below a threshold value or falls within a range of values, a physician treats with physical intervention, such as a stent. If it is determined that blood oxygen delivery is not impeded above a threshold value, the physician explores an alternate treatment option (which may include no treatment or the delipidation process of the present specification). In an embodiment, the threshold value is 80%. In an embodiment, the range of values is 1%-79%.

Subsequently, a physician determines whether both the accumulated lipid-containing degenerative material, covering a lesion/plaque/area/region(s) is in an amount or volume within a predetermined range of percentages and blood oxygen delivery is impeded above a threshold value or within a predetermined range of values. If both threshold conditions are met, the physician treats those areas identified as ischemic areas (FFR below 80%, or within a range of 1% to79%) with a stent implant procedure and subsequently, the remaining areas with the delipidation process of the present specification. If both threshold conditions are not met, then the physician determines if either one of the conditions or neither condition is met and determines an appropriate course of treatment as outlined above.

In an example case, where the analysis from the imaging determines a FFR in the range of 1%-79%, and blockage due to lipids anywhere from 1 to 100%, a physician may decide to physically intervene to improve blood oxygen delivery, as measured by FFR. In an embodiment, the physical intervention is performed by surgically embedding a stent in order to increase the rate of blood flow in the identified atheroma area.

In another example, where the analysis from the imaging determines a FFR in the range of 80%-100%, and blockage due to lipids to be in the range of 20%-70%, the physician may opt for treatment methods that remove or reduce the lipids. In this example, embodiments of the present specification that enable selective modification of HDL particles are utilized.

In yet another example, where the FFR is determined to be less than 80% (in a range of 1% to 79%), and blockage due to lipids is in the range of 20%-70%, the physician may opt to proceed with the surgical process of embedding a stent. It should be appreciated that when a percentage blockage is stated, such as 20%-70%, it means that a cross-sectional area of a vessel is blocked with lipid containing material and that such blockage occupies a range of 20% to 70% of the cross-sectional area of the vessel.

If arteries with lipid-containing atheroma area/volume within a predetermined range of percentages are identified, the patient is then subjected to the delipidation process. In this case, a blood fraction of the patient is obtained. The process of blood fractionation is typically done by filtration, centrifuging the blood, aspiration, or any other method known to persons skilled in the art. Blood fractionation separates the plasma from the blood. In one embodiment, blood is withdrawn from a patient in a volume sufficient to produce about 12 ml/kg of plasma based on body weight. The blood is separated into plasma and red blood cells using methods commonly known to one of skill in the art, such as plasmapheresis. Then the red blood cells are stored in an appropriate storage solution or returned to the patient during plasmapheresis. The red blood cells are preferably returned to the patient during plasmapheresis. Physiological saline is also optionally administered to the patient to replenish volume.

Blood fractionation is known to persons of ordinary skill in the art, and is performed remotely from the method described in context of FIG. 1A. During the fractionation, the blood can optionally be combined with an anticoagulant, such as sodium citrate, and centrifuged at forces approximately equal to 2,000 times gravity. The red blood cells are then aspirated from the plasma. Subsequent to fractionation, the cells are returned to the patient. In some alternate embodiments, Low Density Lipoprotein (LDL) is also separated from the plasma. Separated LDL is usually discarded. In alternative embodiments, LDL is retained in the plasma. In accordance with embodiments of the present specification, obtained blood fraction includes plasma with High Density Lipoprotein (HDL), and may or may not include other protein particles. In embodiments, autologous plasma collected from the patient is subsequently treated via an approved plasmapheresis device. The plasma may be transported using a continuous or batch process.

The blood fraction obtained is mixed with one or more solvents, such as lipid removing agents. In an embodiment, the solvents used include either or both of organic solvents sevoflurane and n-butanol. In embodiments, the plasma and solvent are introduced into at least one apparatus for mixing, agitating, or otherwise contacting the plasma with the solvent. In embodiments, the solvent system is optimally designed such that only the HDL particles are treated to reduce their lipid levels and LDL levels are not affected. The solvent system includes factoring in variables such as solvent employed, mixing method, time, and temperature. Solvent type, ratios and concentrations may vary in this step. Acceptable ratios of solvent to plasma include any combination of solvent and plasma. In some embodiments, ratios used are 2 parts plasma to 1 part solvent, 1 part plasma to 1 part solvent, or 1 part plasma to 2 parts solvent. In an embodiment, when using a solvent comprising 95 parts sevoflurane to 5 parts n-butanol, a ratio of two parts solvent per one part plasma is used. Additionally, in an embodiment employing a solvent containing n-butanol, the present specification uses a ratio of solvent to plasma that yields at least 3% n-butanol in the final solvent/plasma mixture. In an embodiment, a final concentration of n-butanol in the final solvent/plasma mixture is 3.33%. The plasma and solvent are introduced into at least one apparatus for mixing, agitating, or otherwise contacting the plasma with the solvent. The plasma may be transported using a continuous or batch process. Further, various sensing means may be included to monitor pressures, temperatures, flow rates, solvent levels, and the like. The solvents dissolve lipids from the plasma. In embodiments of the present specification, the solvents dissolve lipids to yield treated plasma that contains modified HDL particles with reduced lipid content. The process is designed such that HDL particles are treated to reduce their lipid levels and yield modified HDL particles without destruction of plasma proteins or substantially affecting LDL particles.

Energy is introduced into the system in the form of varied mixing methods, time, and speed. Bulk solvents are removed from the modified HDL particles via centrifugation. In embodiments, any remaining soluble solvent is removed via charcoal adsorption, evaporation, or Hollow Fiber Contractors (HFC) pervaporation. The mixture is optionally tested for residual solvent via use of chromatography (GC), or similar means. The test for residual solvent may optionally be eliminated based on statistical validation.

The treated plasma containing modified HDL particles with reduced lipid content, which was separated from the solvents, is treated appropriately and subsequently returned to the patient. The modified HDL particles are HDL particles with an increased concentration of pre-beta HDL. Concentration of pre-beta HDL is greater in the modified HDL, relative to the original HDL that was present in the plasma before treating it with the solvent. The resulting treated plasma containing the HDL particles with reduced lipid and increased pre-beta concentration is optionally combined with the patient's red blood cells, if the red cells were not already returned during plasmapheresis, and administered to the patient. One route of administration is through the vascular system, preferably intravenously.

In embodiments, the patient is monitored again for changes in the previously monitored atheroma areas and volumes, specifically for lipid-containing degenerative material. Therefore the process is repeated, as described above. In embodiments, the patient is monitored repeatedly within a period of three to six months. The treatment cycle is also repeated at this frequency until the monitoring suggests substantially or completely enhanced cholesterol efflux. In an embodiment, when the atheroma area and volume are monitored to be below threshold, the patient may be considered to have been treated and may not require further repetition of the treatment cycle. In some embodiments, frequency of treatment may vary depending on the volume to be treated and the severity of the condition of the patient.

### Alzheimer's Disease

Alzheimer's disease is determined using results from several tests to arrive at a differential diagnosis. Thus, there is no definitive diagnosis for Alzheimer's disease. In embodiments, treatments and protocols of the present specification are applicable to patients exhibiting pre-symptomatology of AD, in addition to symptoms related to altered global function, cognitive function, activities of daily living (ADL)/functional impairment, and behavior. Thus, patients suffering from AD can be characterized as having early stage (pre-symptomatic)/Stages 1-4, mild, moderate, or severe AD based upon the totality of symptoms.

The following categories may be assigned and are provided for the design and evaluation of benefits throughout the different early stages of AD:
Stage 1 is representative of a class of patients with characteristic pathophysiologic changes of early onset AD but no evidence of clinical impact. These patients are truly asymptomatic with no subjective complaint, functional impairment, or detectable abnormalities on sensitive neuropsychological measures. The characteristic pathophysiologic changes are typically demonstrated by assessment of various biomarker measures.
Stage 2 includes the group of patients with characteristic pathophysiologic changes of early onset AD and subtle detectable abnormalities on sensitive neuropsychological measures, but no functional impairment. The emergence of subtle functional impairment signals a transition to Stage 3.
Stage 3 is representative of a class of patients with characteristic pathophysiologic changes of early onset AD, subtle or more apparent detectable abnormalities on sensitive neuropsychological measures, and mild but detectable functional impairment. The functional impairment in this stage is not severe enough to warrant a diagnosis of overt dementia.
Stage 4 includes a group of patients with overt dementia. This diagnosis is made as functional impairment worsens from that seen in Stage 3. This stage may be refined into additional categories which correspond to mild, moderate, and severe Alzheimer's disease states as described below.
Stages 5, 6, and 7 correspond to increasing degrees of overt dementia and/or functional impairment. As such, stages 5, 6, and 7 correspond to mild, moderate, and severe AD.

In embodiments, a baseline, starting or initial severity level is diagnosed/assessed using at least one physiological diagnostic or advanced medical imaging technique. In some embodiments, a baseline, starting or initial severity level is additionally assessed by at least one cognitive measurement or test. Given the panoply of available neuropsychological tests, a pattern of putatively beneficial effects demonstrated across multiple individual tests may be used to assess impact in early AD or a large magnitude of effect on a single sensitive measure of neuropsychological performance may be used. For example, measuring the level of amyloid peptide (including 40 and 42) may be used to assess a possible treatment benefit.

Differential diagnosis and the assessment of the severity level of Alzheimer's disease may be based on one or more global, cognitive, functional and behavioral measurements, assessments, or tests.

In embodiments, global assessment tests may include assessments such as, but not limited to Clinician's Interview-Based Impression of Change plus caregiver assessment (the CIBIC-plus), and Clinical Dementia Rating-sum of boxes (CDR-SB).

Clinician's Interview-Based Impression of Change plus caregiver input (the CIBIC-plus) is not a single or standardized instrument, such as the ADAS-cog described below. Clinical trials for investigational drugs have used a variety of CIBIC formats, each different in terms of depth and structure. As such, results from a CIBIC-plus reflect clinical experiences from the trial or trials in which it was used and cannot be compared directly with the results of CIBIC-plus evaluations from other clinical trials. By way of example, the CIBIC-plus used in some major trials is a semi-structured instrument that was intended to examine four major areas of patient function: General, Cognitive, Behavioral, and Activities of Daily Living. It represents the assessment of a skilled clinician based upon his/her observations at an interview with the patient, in combination with information supplied by a caregiver familiar with the behavior of the patient over the interval rated. The CIBIC-plus is scored as a seven-point categorical rating, ranging from a score of 1, indicating “markedly improved,” to a score of 4, indicating “no change” to a score of 7, indicating “markedly worse.” The CIBIC-plus has not been systematically compared directly to assessments not using information from caregivers (CIBIC) or other global methods.

Clinical Dementia Rating-sum of boxes (CDR-SB) measures cognitive performance in six areas: memory, orientation, judgment/problem solving, community affairs, home/hobbies, personal care. Each category is scored on five-point scale of impairment (0=none, 0.5=questionable, 1=mild, 2=moderate, 3=severe). The sum of ratings (0-18) provides the overall CDR-SB assessment.

In embodiments, cognitive tests may include assessments such as, but not limited to, the cognitive subscale of the Alzheimer's Disease Assessment Scale (ADAS-cog) and Mini Mental State Examination (MMSE).

The cognitive subscale of the Alzheimer's Disease Assessment Scale (ADAS-cog) is a multi-factor instrument that has been extensively validated in longitudinal cohorts of Alzheimer's disease patients. The ADAS-cog examines selected aspects of cognitive performance including elements of memory, orientation, attention, reasoning, language, and praxis. The ADAS-cog scoring range is from 0 to 70, with higher scores indicating greater cognitive impairment. Elderly adults with normal cognitive functionality may score as low as 0 or 1, but it is not unusual for adults not presenting with typical dementia to score slightly higher.

The Mini Mental State Examination (MMSE) includes 11 questions regarding orientation, memory, concentration, language, and praxis. The scoring scale ranges from 0 to 30, with a higher score indicating lower impairment.

In embodiments, functional tests or tests that assess impairment in activities of daily living, may include assessments such as, but not limited to, Severe Impairment Battery (SIB), Modified Alzheimer's disease Cooperative Study-activities of daily living inventory (ADCS-ADL) and Modified Alzheimer's disease Cooperative Study-activities of daily living inventory for severe Alzheimer's disease (ADCS-ADL-severe), Progressive Deterioration Scale (PDS), Instrumental Activities of Daily Living (IADL), and the Katz Activities of Daily Living (ADL) index.

The Severe Impairment Battery (SIB) assessment is a multi-item instrument and has been validated for the evaluation of cognitive function in patients presenting with moderate to severe dementia. The SIB evaluates selective aspects of cognitive performance, including elements of memory, language, orientation, attention, praxis, visuospatial ability, construction, and social interaction. The SIB scoring range is from 0 to 100, with lower scores indicating greater cognitive impairment.

The Modified Alzheimer's Disease Cooperative Study-Activities of Daily Living inventory (ADCS-ADL) consists of a comprehensive battery of ADL questions used to measure the functional capabilities of patients. Each ADL item is rated from the highest level of independent performance to complete loss. The investigator performs the inventory by interviewing a caregiver familiar with the behavior of the patient. A subset of 19 items, including ratings of the patient's ability to eat, dress, bathe, telephone, travel, shop, and perform other household chores has been validated for the assessment of patients with moderate to severe dementia. The modified ADCS-ADL has a scoring range of 0 to 54, with the lower scores indicative of greater functional impairment.

The Modified Alzheimer's Disease Cooperative Study-Activities of Daily Living Inventory for Severe Alzheimer's Disease (ADCS-ADL-severe) is derived from the Alzheimer's Disease Cooperative Study-Activities of Daily Living Inventory described above, which is a comprehensive battery of ADL questions used to measure the functional capabilities of patients. Each ADL item is rated from the highest level of independent performance to complete loss. The ADCS-ADL-severe is a subset of 19 items, including ratings of the patient's ability to eat, dress, bathe, use the telephone, get around (or travel), and perform other activities of daily living; it has been validated for the assessment of patients with moderate to severe dementia. The ADCS-ADL-severe has a scoring range of 0 to 54, with the lower scores indicative of greater functional impairment. The investigator performs the inventory by interviewing a caregiver, such as a nurse staff member, who is familiar with the overall functional capability of the patient.

The Progressive Deterioration Scale (PDS) examines activities of daily living (ADL) and instrumental ADL in 11 areas, including the extent to which the patient can leave the immediate neighborhood, the use of familiar household implements, involvement in family finances and budgeting, self-care, and routine tasks. The scoring scale ranges from 0 to 100, wherein a higher score indicating better overall functional capability.

The Instrumental Activities of Daily Living (IADL) assessment is used to measure competence in complex ADL, including telephoning, shopping, food preparation, housekeeping, laundering, use of transportation, use of medicine, and the ability to handle money. Each behavioral area is scored 1 or 0. A higher composite score indicates better functional performance.

The Katz Activities of Daily Living (ADL) index is used to assess a patient's ability to perform ADL independently in six functions of bathing, dressing, toileting, transferring, continence, and feeding. Each function is assigned a score of yes or no for independence in that function, whereby each “yes” answer generates one point. A total score of 6 indicates full functional capability while a score of 2 or less is indicative of severe functional impairment.

In embodiments, behavioral and mood tests may include assessments such as, but not limited to, Neuropsychiatric Inventory (NPI) and are employed to determine an extent of depression, anxiety, irritability, and overall mood shifts.

The Neuropsychiatric Inventory (NPI) evaluates 10 items including delusions, hallucinations, dysphoria, anxiety, agitation, euphoria, apathy, irritability, disinhibition, aberrant motor behavior (pacing and rummaging). Two more items may also be assessed, specifically, night-time behavior and changes in appetite and eating behaviors. The frequency of behavioral disturbances are rated on a four-point scale with the severity rated on three-point scale. A higher total score is indicative of more behavioral problems.

In some cases, diagnostic imaging tests are used to determine the accumulation or regional lesions of plaque in the perivascular space. The advanced medical imaging techniques are used to both determine the extent of plaque in the perivascular space and to assess a severity level of Alzheimer's disease. In embodiments, advanced medical imaging techniques, such as, but not limited to, Positron Emission Tomography (PET), Magnetic Resonance Imaging (MRI) and Spinal Fluid Test (Beta Amyloid Fragments), may be used.

A specific Amyloid Positron Emission Tomography (PET) Scan, also referred to as Amyloid PET imaging, represents a potential major advance in an early diagnosis of Alzheimer's disease and/or an assessment of the degree of cognitive impairment. The scan visualizes plaque regions or lesions present in the brain, which are prime suspects in damaging and killing nerve cells in Alzheimer's patients. The scan technique employs radioactive tracers to highlight amyloid protein plaque regions or lesions within the brain, which are a hallmark of Alzheimer's disease. Amyloid PET scanning enables the “illumination” of amyloid plaques on a brain PET scan, enabling accurate detection of plaques in living people. The scan may allow for an earlier diagnosis or assessment of Alzheimer's disease, prior to the presentation of symptomatology.

The practice parameters for the diagnosis and evaluation of dementia, as published by the American Academy of Neurology (AAN), consider structural brain imaging optimal, wherein MRI is one of the appropriate imaging methods. The AAN suggests that neuroimaging may be most useful in patients with dementia characterized by an early onset or an unusual course. Thus, Magnetic Resonance Imaging (MRI) may be considered a preferred neuroimaging examination for diagnosis and assessment of Alzheimer's disease because it allows for accurate measurement of the 3-dimensional (3D) volume of brain structures, and in particular, the size of the hippocampus and related regions. Neuroimaging is widely believed to be generally useful for excluding reversible causes of dementia syndrome, such as normal-pressure hydrocephalus, brain tumors, and subdural hematoma, and for excluding other likely causes of dementia, such as cerebrovascular disease, thereby enabling a differential diagnosis of AD.

Spinal Fluid Test (detection of Beta Amyloid Fragments), is a diagnostic test that requires drawing fluid from the spinal region. Researchers have identified a protein “signature” in the spinal fluid of patients with Alzheimer's disease, which could represent an important advance in its diagnosis. The signature was found in the cerebrospinal fluid (CSF) of 90% of people with a diagnosis of Alzheimer's disease and 72% of people with mild cognitive impairment (MCI)—a disorder that often progresses to Alzheimer's. Researchers measured concentrations of three proteins previously identified as potential biological indicators, or biomarkers, for Alzheimer's and MCI: amyloid-beta, tau, and phospho-tau. Alzheimer's disease was identified in three independent study groups wherein the participants exhibited low levels of the amyloid protein amyloid-beta 1-42, along with high levels of total tau and elevated phospho-tau 181 (P-tau 181).

Apolipoprotein E (ApoE) is a class of proteins involved in the metabolism of fats in the body and is the principal cholesterol carrier in the brain. ApoE is polymorphic, with three major alleles, namely ApoE-ε2, ApoE-ε3, and ApoE-ε4. ApoE-ε2 has an allele frequency of approximately 7% to 8% in the general population. This variant of the apolipoprotein binds poorly to cell surface receptors while ApoE-ε3 and ApoE-ε4 bind relatively well. ApoE-ε2 is associated with both increased and decreased risk for atherosclerosis. Individuals with a ε2/ε2 combination tend to clear dietary fat more slowly and be at greater risk for early vascular disease and the genetic disorder type III hyperlipoproteinemia. ApoE-ε3 has an allele frequency of approximately 80% in the general population. It is considered the “neutral” ApoE genotype of the three. ApoE-ε4 has an allele frequency of approximately 14% in the general population. The ε4 variant is the largest known genetic risk factor for late-onset sporadic Alzheimer's disease (AD).

Although 40-65% of AD patients have at least one copy of the ε4 allele, ApoE-ε4 is not a definitive determinant of the disease; at least one-third of patients with AD are ApoE-ε4 negative and some people with ApoE-ε4 homozygotes never develop the disease. Yet, studies show that those with two ε4 alleles have up to 20 times the risk of developing AD and thus, it can be implicated as at least a contributing factor. There is also evidence that the ApoE-ε2 allele may serve a protective role in AD. Thus, the genotype most at risk for Alzheimer's disease and at an earlier age is ApoE-ε4, ApoE-ε4. Using genotype ApoE-ε3, ApoE-ε3 as a benchmark (allocating a risk factor of 1.0 to the persons who have this genotype), individuals with genotype ApoE-ε4, ApoE-ε4 have a relative risk factor of 14.9 of developing Alzheimer's disease. Individuals with the ApoE-ε3, ApoE-ε4 genotype exhibit a relative risk factor of 3.2, while people with the ε2 allele and the ε4 allele (ApoE-ε2, ApoE-ε4) have a relative risk factor of 2.6. Persons with one copy each of the ε2 allele and the ε3 allele (ApoE-ε2, ApoE-ε3) have a relative risk factor of 0.6, as do persons with two copies of the 2 allele (ApoE-ε2, ApoE-ε2).

While ApoE-ε4 has been found to greatly increase the likelihood that an individual will develop Alzheimer's disease, it should be noted that persons with any combination of independent risk factors, such as but not limited to different levels of certain ApoE alleles as described above, high overall serum total cholesterol levels, and high blood pressure have an amplified risk of developing AD at some point in their lifetime. Accordingly, research has suggested that lowering serum cholesterol levels may reduce a person's risk for Alzheimer's disease, even if they have two ApoE-ε4 alleles, thus reducing the risk from nine or ten times the odds of developing AD down to just two times the odds. Women are more likely to develop AD than men across most ages and persons with at least one ε4 allele have significantly more neurological dysfunction than men.

In aspects of the present specification, a treated plasma that contains modified HDL particles with reduced lipid content is delivered to the patient via infusion therapy. The process is designed such that HDL particles are treated to reduce their lipid levels and yield modified HDL particles without destruction of plasma proteins or substantially affecting LDL particles.

The HDL lipoprotein particles are comprised of ApoA-I, phospholipids and cholesterol. Persons of ordinary skill in the art would appreciate that Apolipoprotein A-I (ApoA-I) particles comprise of two sub-fractions, pre-β HDL and α-HDL, which have pre-beta and alpha electrophoretic mobility, respectively. Thus, pre-β HDL 645 represents ApoA-I molecules complexed with phospholipids.

In aspects of the present specification, a treated plasma that contains modified HDL particles with reduced lipid content is delivered to the patient via infusion therapy. In an embodiment, the modified high density lipoproteins have a concentration of alpha high density lipoproteins in addition to the pre-beta high density lipoproteins from the blood fraction prior to mixing.

In aspects of the present specification, isolated pre-β HDL particles are infused into the patient's blood stream to bind to beta amyloid particles and clear the cerebral perivascular pathway.

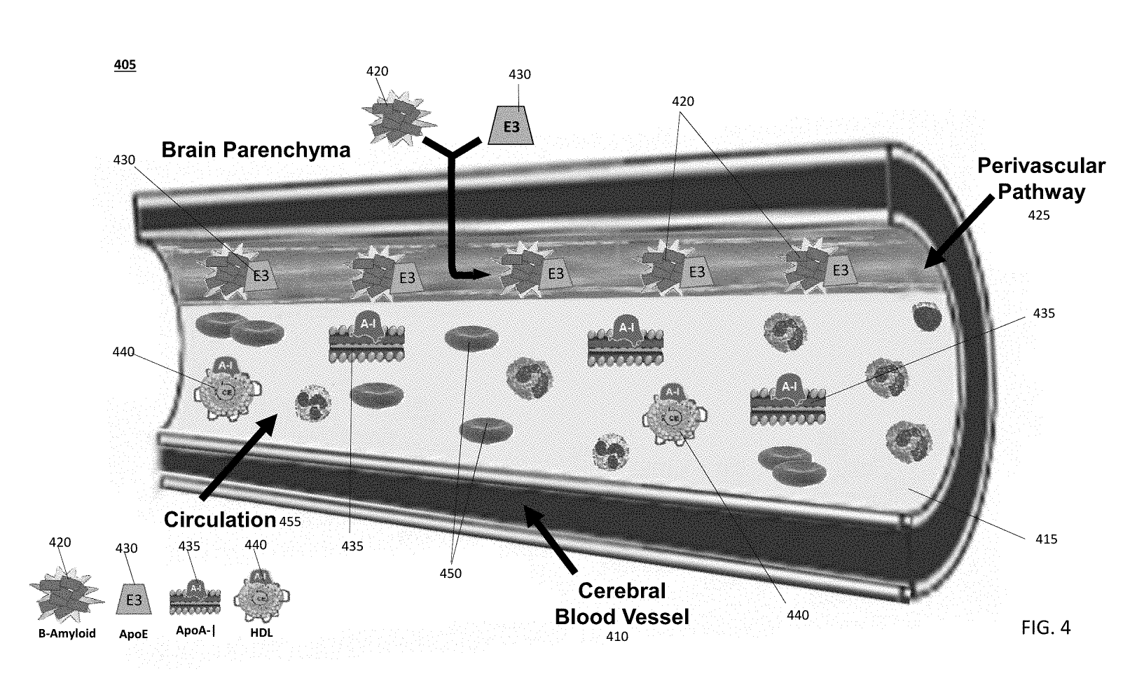

FIG. 4 is a longitudinal transverse cross-sectional view 405 of a cerebral blood vessel 410 illustrating removal of beta amyloid by transport along the cerebral lymphatic perivascular pathway, in accordance with an embodiment of the present specification. As shown, blood circulates through the lumen 415 of the vessel 410 while Interstitial Fluid (ISF) and solutes, including beta amyloid (Aβ) 420, are eliminated from the brain through the perivascular drainage pathway 425, which is, effective, the lymphatic drainage of the brain. The ε3 allele 430 binds to beta amyloid particles 420, forming modified ε3 particles, and thereby transporting beta amyloid particles 420 from the brain along the perivascular drainage pathway 425. Also shown are Apolipoprotein A-I (ApoA-I) particles 435 and HDL particles 440 as part of the blood circulation 455 through the lumen 415 along with other particles such as, for example, red blood cells 450. AD is, in some cases, characterized by build-ups of aggregates of the peptide beta-amyloid in the cerebral lymphatic perivascular pathways. As illustrated in Table A, in AD patients the distribution of ε2, ε3 and ε4 alleles is approximately 4%, 60% and 37%, respectively. The isoform ApoE-ε4 is not as effective as the alleles at promoting clearance of beta amyloid from the cerebral perivascular drainage pathways. Thus, a skewed abundance of ε4 allele is associated with increased vulnerability to AD in individuals with that gene variation and in AD patients is also associated with an increase in the severity of AD and loss of cognitive function.

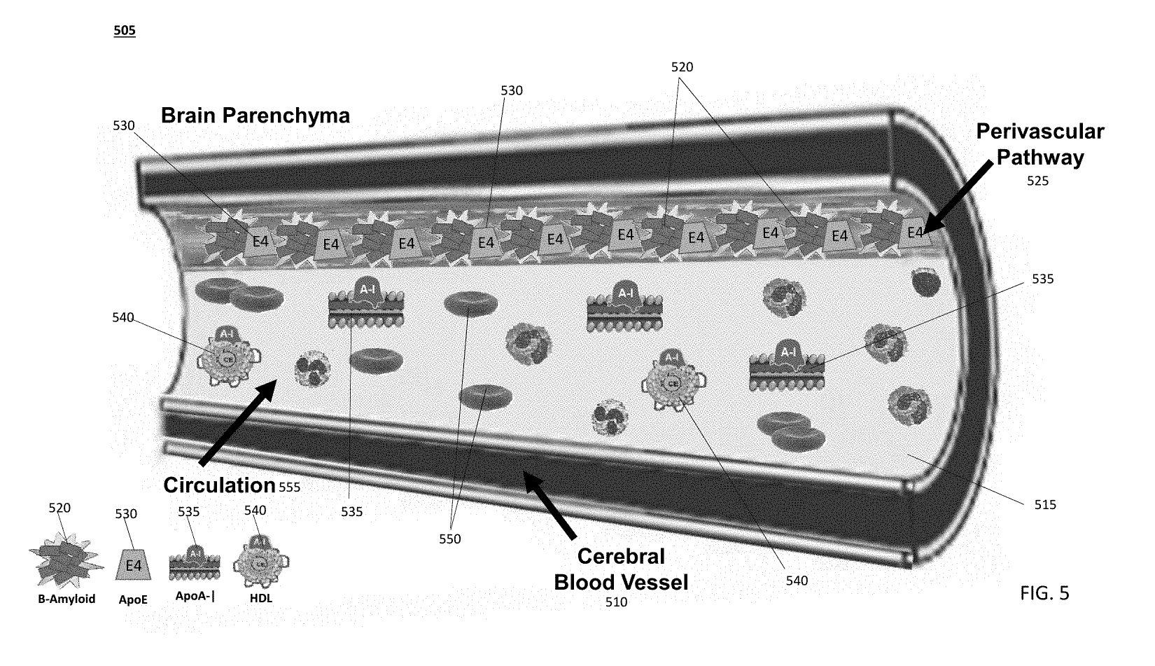

FIG. 5 is a longitudinal transverse cross-sectional view 505 of a cerebral blood vessel 510 illustrating amyloid accumulation in cerebral lymphatic perivascular pathways of individuals with an increased presence of the ε4 allele, in accordance with an embodiment of the present specification. As shown, blood circulates through the lumen 515 of the vessel 510 while beta amyloid (Aβ) particles 520 are accumulated in the cerebral perivascular pathway 525 due to an increased presence of ε4 particles 530. Thus, beta amyloid 520 is deposited in the walls of the blood vessel 510 as cerebral amyloid angiopathy (CAA). CAA in AD reflects a failure of elimination of amyloid-beta (Aβ) from the brain along perivascular lymphatic drainage pathways 525. Failure of elimination of beta amyloid along perivascular pathways may coincide with a reduction in enzymatic degradation of beta amyloid, reduced absorption of beta amyloid into the blood and stiffening of blood vessel walls. Also shown are ApoA-I particles 535 and HDL particles 540 as part of the blood circulation 555 through the lumen 515 along with other particles such as, for example, red blood cells 550.

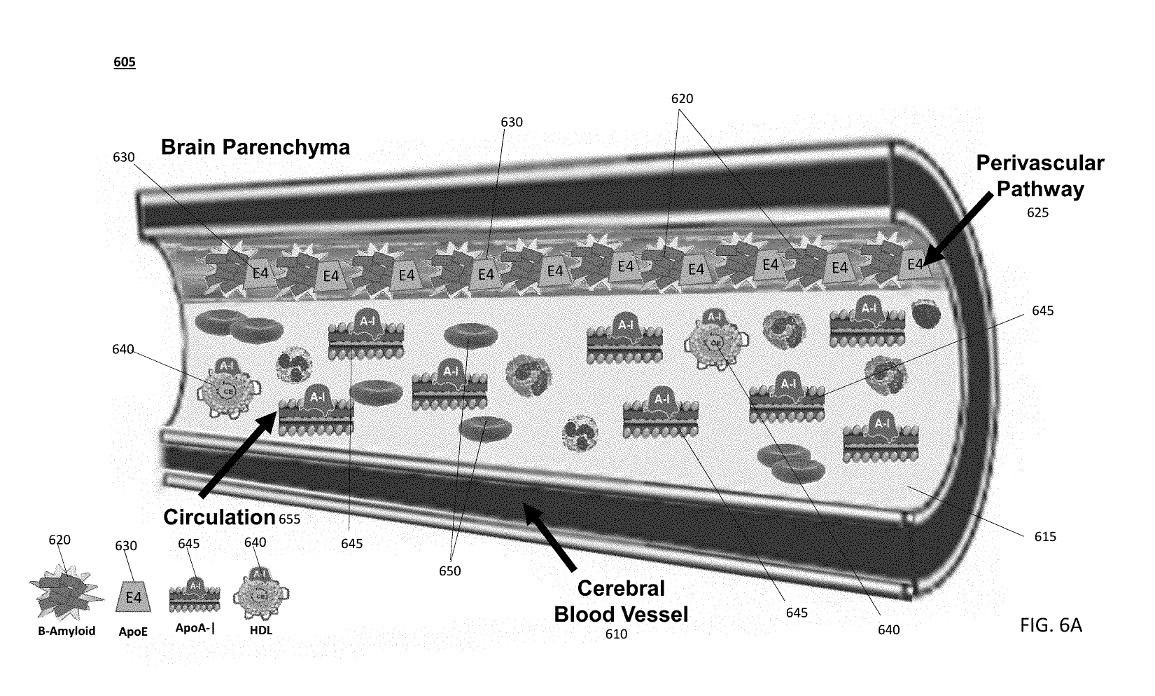

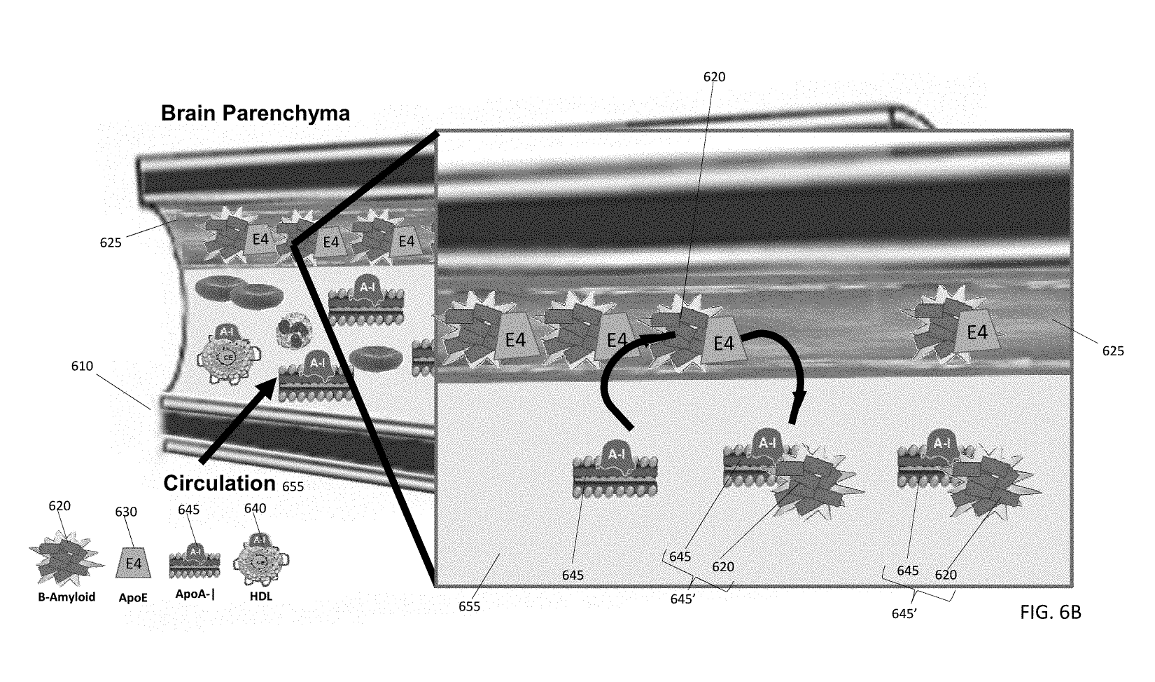

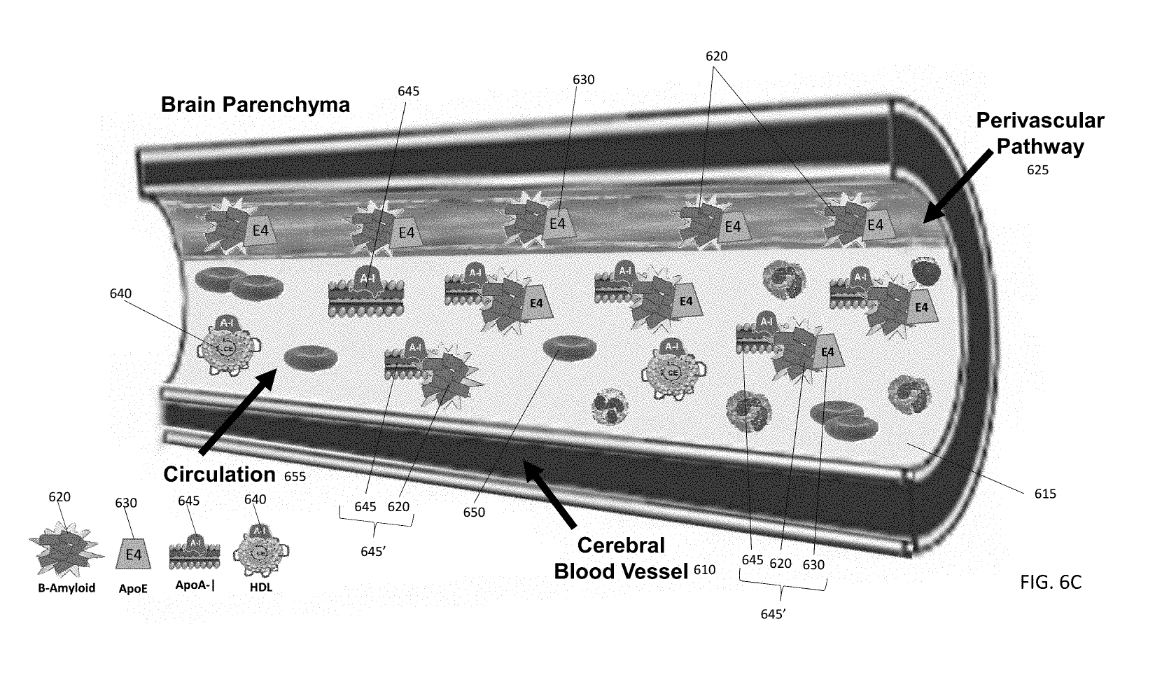

FIG. 6A is a longitudinal transverse cross-sectional view 605 of a cerebral blood vessel 610 of an AD patient being treated for cerebral amyloid angiopathy (CAA), in accordance with an embodiment of the present specification. As shown, blood circulates through the lumen 615 of the vessel 610 while beta amyloid (Aβ) particles 620 accumulate in the cerebral perivascular pathway 625 along with a high presence of ε4 particles 630, thereby essentially blocking pathway 625. In accordance with an aspect of the present specification, treated plasma or isolated pre-β HDL particles 645 are infused into the patient's blood stream 655 to bind to beta amyloid particles 620 and clear the cerebral perivascular pathway 625. Pre-β HDL 645 represents ApoA-I molecules complexed with phospholipids.

To generate and subsequently infuse the patient with treated plasma or with a solution containing an increased concentration of isolated pre-β HDL 645, a blood fraction is obtained. The process of blood fractionation is typically done by filtration, centrifuging the blood, aspiration, or any other method known to persons skilled in the art. Blood fractionation separates the plasma from the blood. In one embodiment, blood is withdrawn from a patient in a volume sufficient to produce about 12 ml/kg of plasma based on body weight. The blood is separated into plasma and red blood cells using methods commonly known to one of skill in the art, such as plasmapheresis. Then the red blood cells are stored in an appropriate storage solution or returned to the patient during plasmapheresis. The red blood cells are preferably returned to the patient during plasmapheresis. Physiological saline is also optionally administered to the patient to replenish volume.

In some alternate embodiments, Low Density Lipoprotein (LDL) is also separated from the plasma. Separated LDL is usually discarded. In alternative embodiments, LDL is retained in the plasma. In accordance with embodiments of the present specification, the resultant blood fraction includes plasma with HDL, and may or may not include other protein particles.

In one embodiment, the process of blood fractionation is performed by withdrawing blood from the patient presenting with AD, and who is being treated by the physician. In an alternative embodiment, the process of blood fractionation is performed by withdrawing blood from a person other than the patient. Therefore, the plasma obtained as a result of the blood fractionation process may be either autologous or non-autologous.

In an optional embodiment, the autologous or non-autologous plasma obtained is subjected to a delipidation process as described in greater detail above with respect to FIG. 1A but repeated briefly herein. The resultant blood fraction is mixed with one or more solvents, such as lipid removing agents. In an embodiment, the solvents used include either or both of organic solvents sevoflurane and n-butanol. In embodiments, the plasma and solvent are introduced into at least one apparatus for mixing, agitating, or otherwise contacting the plasma with the solvent. In embodiments, the solvent system is optimally designed such that only the HDL particles are treated to reduce their lipid levels and LDL levels are not affected. The solvent system includes factoring in variables such as solvent employed, mixing method, time, and temperature. Solvent type, ratios and concentrations may vary in this step. The plasma and solvent are introduced into at least one apparatus for mixing, agitating, or otherwise contacting the plasma with the solvent. The plasma may be transported using a continuous or batch process. The solvents dissolve lipids from the plasma. In embodiments of the present specification, the solvents dissolve lipids to yield treated plasma that contains modified HDL particles with reduced lipid content. The process is designed such that HDL particles are treated to reduce their lipid levels and yield modified HDL particles without destruction of plasma proteins or substantially affecting LDL particles. The resultant treated plasma containing modified HDL particles with reduced lipid content, which was separated from the solvents, is treated appropriately and may subsequently returned to the patient in an embodiment.

In an optional embodiment, the resultant fluid containing modified HDL particles is further processed, in a second stage, to separate or to isolate pre-β HDL particles. In an embodiment, the second stage occurs in a separate and discrete area from the delipidation process. In an alternate embodiment, the second stage processing occurs in-line with the delipidation system, whereby the system may be connected to an affinity column sub-system or ultracentrifugation sub-system. The resultant separated pre-β HDL particles may then be introduced to the bloodstream of the patient as described below.

FIG. 6A illustrates a presence of non-modified HDL particles 640 in the blood stream 655 along with other particles such as, for example, red blood cells 650. The modified HDL particles may be HDL particles with an increased concentration of pre-β HDL particles 645. Concentration of pre-β HDL 645 is greater in the modified HDL, relative to the original HDL that was present in the plasma before treating it with the solvent. The resulting treated plasma containing the HDL particles with reduced lipid and increased pre-β concentration is optionally combined with the patient's red blood cells, if the red cells were not already returned during plasmapheresis, and administered to the patient. One route of administration is through the vascular system, preferably intravenously, such as via infusion therapy.

FIG. 6B illustrates a mechanism of removal of beta amyloid molecules 620 by infused pre-β HDL particles 645 within the blood vessel 610 of an AD patient, in accordance with an embodiment of the present specification. As shown, with increased concentration of pre-β HDL particles 645 in the patient's blood stream 655, a relatively higher number of pre-β HDL particles 645 are available to bind to and pull out beta-amyloid particles 620 from the perivascular pathway 625. The pre-β HDL particles 645 in the blood stream 655 enter the perivascular pathway 1025 and bind with beta-amyloid particles 620 to form modified pre-β HDL particles 645′ that re-enter the blood stream 655.

FIG. 6C shows a plurality of modified pre-β HDL particles 645′ flowing in the blood stream 655 (in the lumen 615 of the blood vessel 610) and serving to transport the bound beta amyloid 620 to the liver for degradation and subsequent excretion. In some embodiments, the pre-β HDL particles 6145 also pull the ε4 particles 630 along with the beta amyloid molecules 620 from the perivascular pathway 625. In such embodiments, the modified pre-β HDL particles 645′ are pre-β HDL 645 binding both beta amyloid 620 and ε4 630. Thus, the infused isolated pre-β HDL particles 1145 initiate reverse cholesterol, specifically beta amyloid 620, transport process from the cerebral perivascular pathways 625 to liver. Also seen in FIG. 6C are non-modified HDL particles 640 in the blood stream 655 along with other particles such as, for example, red blood cells 650.

### Therapeutic Protocols for Administering Modified HDL Particles

In accordance with aspects of the present specification, treated plasma containing modified HDL particles with reduced lipid and/or increased pre-β concentration is administered to a patient in accordance with a plurality of therapeutic protocols. In some embodiments, therapy is based on a level of severity of AD, as described above. In various embodiments, the plurality of therapy protocols comprises at least one or any combination of a plurality of therapeutic parameters such as, but not limited to:
Dosing range: 1 mg/kg to 250 mg/kg, and any increment therein, where a specific fixed dose may be calculated based on one or both of a patient's weight and the severity of the disease state.
Dosing volume: the average dosing volume is dependent upon the dose (in mg/kg) and the concentration of the product to be infused into the patient (treated plasma containing modified HDL particles or isolated pre-beta particles). In embodiments, the volume that is returned to the patient is substantially equal to the volume that was removed from the patient prior to the delipidation process. In embodiments, the volume that is returned to the patient is a concentrated volume. In embodiments, the volume delivered to a patient via infusion therapy is dependent upon the preparation of the product, whether it is treated plasma or concentrated, isolated pre-beta and the overall solubility of that product in a buffer or saline.
Dosing rate: the dose is provided via infusion therapy. It should be noted herein that the rate of infusion is the normal infusion rate for intravenous therapy, or 999 mL/hour and is thus dependent on overall volume and concentration. In an embodiment, the time of infusion ranges from one hour to eight hours.
Frequency or cycle of treatment: daily, weekly, monthly and annually
Duration or course of therapy: at least one day to at least one year

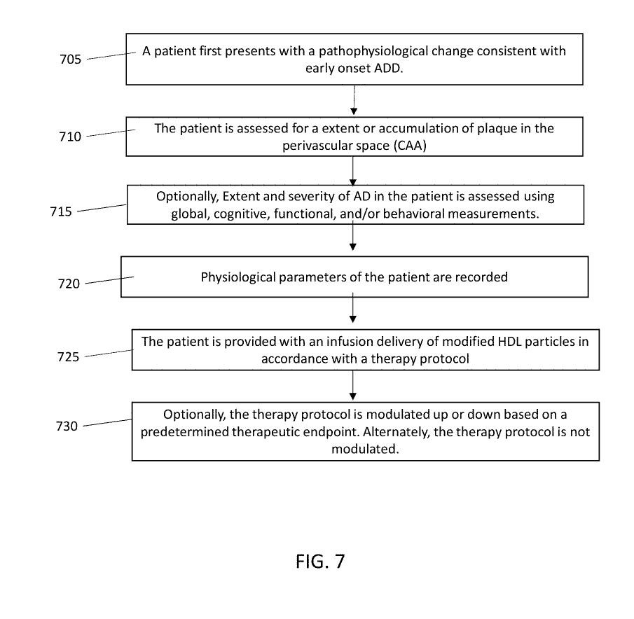

FIG. 7 is a flowchart describing a plurality of exemplary steps of a therapy protocol for treating an AD patient, in accordance with an embodiment of the present specification. At step 705, a patient first presents with a pathophysiological change that is consistent with early onset AD. Any of the aforementioned diagnostic techniques may be used in this step. In embodiments various biomarkers may be used to determine the pathophysiological change. For example, measuring the level of amyloid peptide (including 40 and 42) may be used to assess the extent of a pathophysiological change characteristic of AD. In an embodiment, the patient may present with cerebral amyloid angiopathy (CAA) as detected using a diagnostic imaging technique. At step 710, a patient who is diagnosed with CAA is monitored to determine an extent of accumulation of plaque in the perivascular space, via at least one diagnostic procedure. In embodiments, advanced medical imaging techniques, such as, but not limited to, Positron Emission Tomography (PET), Magnetic Resonance Imaging (MRI) and Spinal Fluid Test (Beta Amyloid Fragments), may be used.

In embodiments, at optional step 715, the diagnosis and severity level of AD in the patient are additionally assessed based on one or more global, cognitive, functional, and behavioral measurements or tests, as described above.

In embodiments, global assessment tests may include assessments such as, but not limited to Clinician's Interview-Based Impression of Change plus caregiver assessment (the CIBIC-plus), and Clinical Dementia Rating-sum of boxes (CDR-SB).

In embodiments, cognitive tests may include assessments such as, but not limited to, cognitive subscale of the Alzheimer's Disease Assessment Scale (ADAS-cog), and Mini Mental State Examination (MMSE).

In embodiments, functional tests may include assessments such as, but not limited to, severe impairment battery (SIB), modified Alzheimer's disease cooperative study—activities of daily living inventory (ADCS-ADL) and modified Alzheimer's disease cooperative study activities of daily living inventory for severe Alzheimer's disease (ADCS-ADL-severe), Progressive Deterioration Scale (PDS), Instrumental Activities of Daily Living (IADL), and Katz activities of daily living (ADL) index.

In embodiments, behavioral and mood tests may include assessments such as, but not limited to, Neuropsychiatric Inventory (NPI).

At step 720, one or more physiological parameters of the patient are recorded. In embodiments, the one or more physiological parameters are those that may be incidental to determining one or more therapy parameters. For example, the patient's weight is recorded to determine a dosing range for the patient. It should be appreciated that the physiological parameters may be first recorded prior to step 705.

At step 725, the patient is infused with modified HDL particles in accordance with a therapy protocol. In an optional embodiment, a blood fraction is withdrawn from the patient presenting with the CAA. In an alternative embodiment, a blood fraction is obtained withdrawing blood from a person other than the patient. Therefore, the plasma obtained as a result of the blood fractionation process may be either autologous or non-autologous. The blood fraction is subsequently treated, using the delipidation process described above to obtain treated plasma containing modified HDL particles. The treated plasma is optionally processed further to generate a product with an increased concentration of isolated pre-β HDL.

In an embodiment, the therapy protocol comprises an infusion delivery of modified HDL particles or a concentrated volume of isolated pre-beta particles over a period ranging from 1 hour to 8 hours, and any increment therein, depending upon the concentration of the therapeutic product to be delivered. In some embodiments, the dose ranges from 1 mg/kg to 250 mg/kg, and any increment therein, and is administered at an infusion delivery rate of 999 mL/hour+/−100 ml/hour or a rate deemed more appropriate for the patient. In embodiments, the treatment is repeated at specified frequency or cycle of treatment depending upon a course of therapy. In some embodiments, the frequency or cycle of administering the treatment may range from once a week, twice a week, three times per week, daily, once a month, twice a month, three times per month, to at least once in three, six, nine or twelve months. In some embodiments, the course of therapy may range from at least one day, at least one week, at least one month to at least one year.

In an alternate embodiment, the therapy protocol comprises at least one, and up to three, seven or ten treatments every three, six, nine or twelve months for an annual course of therapy. In some embodiments, the at least one treatment may comprise a continuous infusion (IV) of modified HDL particles over a predetermined time period at a rate of 999 mL/hour.

At optional step 730, the therapy protocol may be titrated or modulated up or down based on a therapeutic endpoint. In embodiments, one or more intra-treatment severity level assessments are made using diagnostic and/or cognitive procedures/tests. The one or more intra-treatment severity level assessments are made at predetermined points in time during the course of therapy. If the intra-treatment severity level assessments show a delay in the onset of additional symptoms, a halting in the worsening of symptoms, or an improvement in the patient's condition, it is considered to be of therapeutic benefit. In embodiments, when therapeutic benefit is shown, the therapeutic amount may be titrated down wherein parameters such as, but not limited to, the dose range, frequency or cycle of treatment and/or course of therapy may be reduced. Alternately, the therapy protocol may be titrated up depending on various factors. Still alternately, if the intra-treatment severity level assessments show or do not show improvement in the patient's condition, the therapy protocol is not modulated.

By way of example, for an early onset AD patient weighing 100 kg, where a dosage is determined to be 15 mg/kg, that patient will receive a dose of 1.5 g. It should be noted that if the patient presents with mild, moderate or severe AD, that dosage may be increased. The overall volume delivered to the patient via infusion therapy depends on the therapeutic product that is solubilized in a buffer or saline. For example, if the therapeutic product is autologous treated plasma, then the patient will receive a volume of therapeutic product equivalent to the volume that was extracted from the patient. If the therapeutic product is non-autologous treated plasma, the patient may receive a volume of 1 L as one example. If the therapeutic product is non-autologous isolated, concentrated pre-beta particles, the volume may be much lower.

### Therapeutic Endpoints or Objectives

In various embodiments, an AD patient's baseline, starting or initial severity level is diagnosed/assessed and categorized as, one of early onset, mild, moderate or severe as described above. The baseline, starting or initial severity level refers to the severity of AD before the patient is treated with the modified HDL and/or isolated pre-β HDL therapy of the present specification.

In embodiments, the baseline, starting or initial severity level is diagnosed/assessed using at least one physiological diagnostic or advanced medical imaging technique. In some embodiments, the baseline, starting or initial severity level is additionally assessed by at least one global, cognitive, functional, behavioral measurement or test.

In an embodiment, a therapeutic benefit is recognized when a patient is able to maintain or be stabilized in their current state when treated with a therapy protocol of the present specification.

In an embodiment, a therapeutic benefit is recognized when a patient maintains/stabilizes symptoms when treated with a therapy protocol of the present specification when compared to a placebo.

In an embodiment, a therapeutic benefit is recognized when a patient shows a delay or halting of worsening of symptoms when treated with a therapy protocol of the present specification when compared to a placebo.

In an embodiment, a therapeutic benefit is recognized when a patient shows a delay in the rate of progression of symptoms when treated with a therapy protocol of the present specification when compared to a placebo.

In an embodiment, a therapeutic benefit is recognized when a patient shows an improvement in symptoms when treated with a therapy protocol of the present specification when compared to a placebo.

In some embodiments, after at least one treatment session or determinable time period at the end of which an effect of said at least one treatment session is amenable to measurement, the patient experiences a decrease in the accumulation of amyloid plaque in the perivascular space.

Following are a plurality of non-limiting, exemplary therapeutic endpoints with reference to the baseline, starting or initial severity level:

In embodiments, after at least one treatment session or determinable time period at the end of which an effect of said at least one therapy treatment is amenable to measurement, the rate of progression, level or amount of a patient's physiological and/or cognitive parameter, is unchanged relative to the rate, level or amount of that patient's physiological and/or cognitive parameter before therapy treatment.

In embodiments, after at least one treatment session or determinable time period at the end of which an effect of said at least one therapy treatment is amenable to measurement, the rate of progression, level or amount of a patient's physiological and/or cognitive parameter, is delayed relative to the rate, level or amount of that patient's physiological and/or cognitive parameter before therapy treatment.

In embodiments, after at least one treatment session or determinable time period at the end of which an effect of said at least one therapy treatment is amenable to measurement, the rate of progression, level or amount of a patient's physiological and/or cognitive parameter, is modified relative to the rate of progression, level or amount of that patient's physiological and/or cognitive parameter before therapy treatment.

In embodiments, after at least one treatment session or determinable time period at the end of which an effect of said at least one therapy treatment is amenable to measurement, the rate of progression, level or amount of that patient's physiological and/or cognitive parameter is improved relative to the rate of progression, level or amount of that patient's physiological and/or cognitive parameter before therapy treatment.

In some embodiments, after at least one treatment session or determinable time period at the end of which an effect of said at least one treatment session is amenable to measurement, the patient experiences an improvement of the ADAS-cog score indicative of an improvement in, or stabilization of, AD-related symptoms.

In some embodiments, after at least one treatment session or determinable time period at the end of which an effect of said at least one treatment session is amenable to measurement, the patient experiences an improvement of the CIBIC-plus score indicative of an improvement in, or stabilization of, AD-related symptoms.

In some embodiments, after at least one treatment session or determinable time period at the end of which an effect of said at least one treatment session is amenable to measurement, the patient experiences an improvement of the SIB score indicative of an improvement in, or stabilization of, AD-related symptoms.

In some embodiments, after at least one treatment session or determinable time period at the end of which an effect of said at least one treatment session is amenable to measurement, the patient experiences an improvement of the ADCS-ADL score indicative of an improvement in, or stabilization of, AD-related symptoms.

In some embodiments, after at least one treatment session or determinable time period at the end of which an effect of said at least one treatment session is amenable to measurement, the patient experiences an improvement of the ADCS-ADL-severe score indicative of an improvement in, or stabilization of, AD-related symptoms.

In some embodiments, after at least one treatment session or determinable time period at the end of which an effect of said at least one treatment session is amenable to measurement, the patient experiences an improvement of any one of the global, cognitive, functional, or behavioral test scores indicative of an improvement in, or stabilization of, AD-related symptoms.

In some embodiments, after at least one treatment session or determinable time period at the end of which an effect of said at least one treatment session is amenable to measurement, the patient experiences a decrease in the accumulation of amyloid plaque in the perivascular space indicative of an improvement or stabilization of AD-related symptoms.

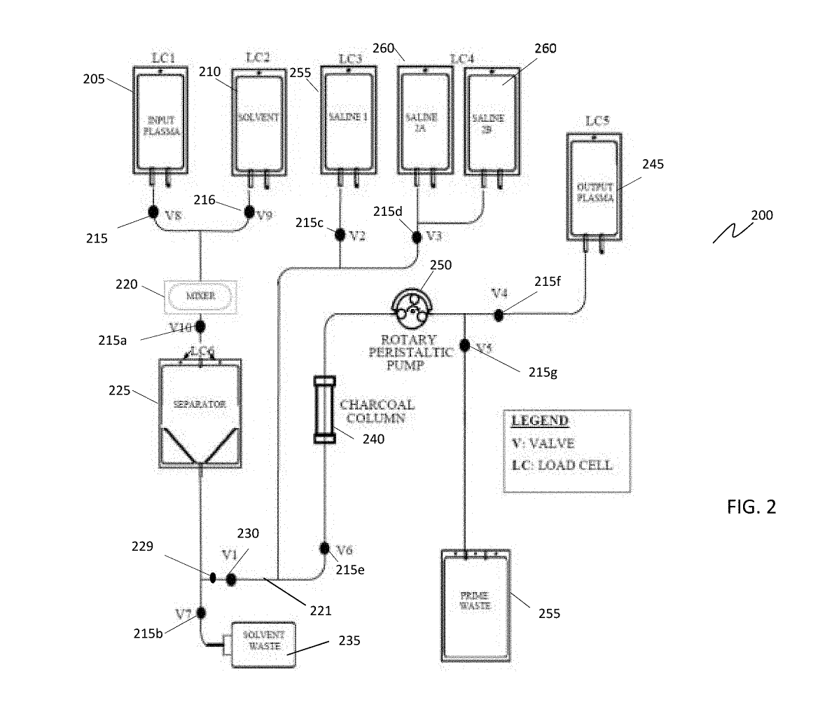

FIG. 2 illustrates an exemplary embodiment of a system and its components used to achieve the methods of the present specification. The figure depicts an exemplary basic component flow diagram defining elements of the HDL modification system 200. Embodiments of the components of system 200 are utilized after obtaining a blood fraction from a patient or another individual (donor). The plasma, separated from the blood is brought in a sterile bag to system 200 for further processing. The plasma may be separated from blood using a known plasmapheresis device. The plasma may be collected from the patient into a sterile bag using standard apheresis techniques. The plasma is then brought in the form of a fluid input to system 200 for further processing. In embodiments, system 200 is not connected to the patient at any time and is a discrete, stand-along system for delipidating plasma. The patient's plasma is processed by system 200 and brought back to the patient's location to be reinfused back into the patient. In alternate embodiments, the system may be a continuous flow system that is connected to the patient in which both plasmapheresis and delipidation are performed in an excorporeal, parallel system and the delipidated plasma product is returned to the patient.

A fluid input 205 (containing blood plasma) is provided and connected via tubing to a mixing device 220. A solvent input 210 is provided and also connected via tubing to mixing device 220. In embodiments, valves 215, 216 are used to control the flow of fluid from fluid input 205 and solvent from solvent input 210 respectively. It should be appreciated that the fluid input 205 contains any fluid that includes HDL particles, including plasma having LDL particles or devoid of LDL particles, as discussed above. It should further be appreciated that solvent input 210 can include a single solvent, a mixture of solvents, or a plurality of different solvents that are mixed at the point of solvent input 210. While depicted as a single solvent container, solvent input 210 can comprise a plurality of separate solvent containers. Embodiments of types of solvents that may be used are discussed above.

Mixer 220 mixes fluid from fluid input 205 and solvent from solvent input 210 to yield a fluid-solvent mixture. In embodiments, mixer 220 is capable of using a shaker bag mixing method with the input fluid and input solvent in a plurality of batches, such as 1, 2, 3 or more batches. An exemplary mixer is a Barnstead Labline orbital shaker table. In alternative embodiments, other known methods of mixing are utilized. Once formed, the fluid-solvent mixture is directed, through tubing and controlled by at least one valve 215a, to a separator 225. In an embodiment, separator 225 is capable of performing bulk solvent separation through gravity separation in a funnel-shaped bag.

In separator 225, the fluid-solvent mixture separates into a first layer and second layer. The first layer comprises a mixture of solvent and lipid that has been removed from the HDL particles. The first layer is transported through a valve 215b to a first waste container 235. The second layer comprises a mixture of residual solvent, modified HDL particles, and other elements of the input fluid. One of ordinary skill in the art would appreciate that the composition of the first layer and the second layer would differ based upon the nature of the input fluid. Once the first and second layers separate in separator 225, the second layer is transported through tubing to a solvent extraction device 240. In an embodiment, a pressure sensor 229 and valve 230 is positioned in the flow stream to control the flow of the second layer to solvent extraction device 240.

The opening and closing of valves 215, 216 to enable the flow of fluid from input containers 205, 210 may be timed using mass balance calculations derived from weight determinations of the fluid inputs 205, 210 and separator 225. For example, the valve 215b between separator 225 and first waste container 235 and valve 230 between separator 225 and solvent extraction device 240 open after the input masses (fluid and solvent) substantially balances with the mass in separator 225 and a sufficient period of time has elapsed to permit separation between the first and second layers. Depending on what solvent is used, and therefore which layer settles to the bottom of separator 225, either valve 215b between separator 225 and first waste container 235 is opened or valve 230 between separator 225 and solvent extraction device 240 is opened. One of ordinary skill in the art would appreciate that the timing of the opening is dependent upon how much fluid is in the first and second layers and would further appreciate that it is preferred to keep valve 215b between separator 225 and first waste container 235 open just long enough to remove all of the first layer and some of the second layer, thereby ensuring that as much solvent as possible has been removed from the fluid being sent to solvent extraction device 240.

In embodiments, an infusion grade fluid (“IGF”) may be employed via one or more inputs 260 which are in fluid communication with the fluid path 221 leading from separator 225 to solvent extraction device 240 for priming. In an embodiment, saline is employed as the infusion grade priming fluid in at least one of inputs 260. In an embodiment, 0.9% sodium chloride (saline) is employed. In other embodiments, glucose may be employed as the infusion grade priming fluid in any one of inputs 260.

In embodiments, a glucose input 255 and one or more saline inputs 260 are in fluid communication with the fluid path 221 leading from separator 225 to solvent extraction device 240. A plurality of valves 215c and 215d are also be incorporated in the flow stream from glucose input 255 and saline input 260 respectively, to the tubing providing the flow path 221 from separator 225 to solvent extraction device 240. IGF such as saline and/or glucose are incorporated into embodiments of the present specification in order to prime solvent extraction device 240 prior to operation of the system. In embodiments, saline is used to prime most of the fluid communication lines and solvent extraction device 240. If priming is not required, the IGF inputs are not employed. Where such priming is not required, the glucose and saline inputs are not required. Also, one of ordinary skill in the art would appreciate that the glucose and saline inputs can be replaced with other primers if required by the solvent extraction device 240 requires it.

In some embodiments, solvent extraction device 240 is a charcoal column designed to remove the specific solvent used in solvent input 210. An exemplary solvent extraction device 240 is an Asahi Hemosorber charcoal column, or the Bazter/Gambro Adsorba 300C charcoal column or any other charcoal column that is employed in blood hemoglobin perfusion procedures. A pump 250 is used to move the second layer from separator 225, through solvent extraction device 240, and to an output container 245. In embodiments, pump 250 is a rotary peristaltic pump, such as a Masterflex Model 77201-62.

The first layer is directed to waste container 235 that is in fluid communication with separator 225 through tubing and at least one valve 215b. Additionally, other waste, if generated, can be directed from the fluid path connecting solvent extraction device 240 and output container 245 to a second waste container 255. Optionally, in an embodiment, a valve 215f is included in the path from the solvent extraction device 240 to the output container 245. Optionally, in an embodiment, a valve 215g is included in the path from the solvent extraction device 240 to the second waste container 255.

In an embodiment of the present specification, gravity is used, wherever practical, to move fluid through each of the plurality of components. For example, gravity is used to drain input plasma 205 and input solvent 210 into mixer 220. Where mixer 220 comprises a shaker bag and separator 225 comprises a funnel bag, fluid is moved from the shaker bag to the funnel bag and, subsequently, to first waste container 235, if appropriate, using gravity.

In an additional embodiment, not shown in FIG. 2, the output fluid in output container 245 is subjected to a solvent detection system, or lipid removing agent detection system, to determine if any solvent, or other undesirable component, is in the output fluid. In embodiments, a solvent sensor is only employed in a continuous flow system. In one embodiment, the output fluid is subjected to sensors that are capable of determining the concentrations of solvents introduced in the solvent input, such as n-butanol or di-isopropyl ether. The output fluid is returned to the bloodstream of the patient and the solvent concentrations must be below a predetermined level to carry out this operation safely. In embodiments, the sensors are capable of providing such concentration information on a real-time basis and without having to physically transport a sample of the output fluid, or air in the headspace, to a remote device. The resultant separated modified HDL particles are then introduced to the bloodstream of the patient.

In one embodiment, molecularly imprinted polymer technology is used to enable surface acoustic wave sensors. A surface acoustic wave sensor receives an input, through some interaction of its surface with the surrounding environment, and yields an electrical response, generated by the piezoelectric properties of the sensor substrate. To enable the interaction, molecularly imprinted polymer technology is used. Molecularly imprinted polymers are plastics programmed to recognize target molecules, like pharmaceuticals, toxins or environmental pollutants, in complex biological samples. The molecular imprinting technology is enabled by the polymerization of one or more functional monomers with an excess of a crosslinking monomer in presence of a target template molecule exhibiting a structure similar to the target molecule that is to be recognized, i.e. the target solvent.

The use of molecularly imprinted polymer technology to enable surface acoustic wave sensors can be made more specific to the concentrations of targeted solvents and are capable of differentiating such targeted solvents from other possible interferents. As a result, the presence of acceptable interferents that may have similar structures and/or properties to the targeted solvents would not prevent the sensor from accurately reporting existing respective solvent concentrations.

Alternatively, if the input solvent comprises certain solvents, such as n-butanol, electrochemical oxidation could be used to measure the solvent concentration. Electrochemical measurements have several advantages. They are simple, sensitive, fast, and have a wide dynamic range. The instrumentation is simple and not affected by humidity. In one embodiment, the target solvent, such as n-butanol, is oxidized on a platinum electrode using cyclic voltammetry. This technique is based on varying the applied potential at a working electrode in both the forward and reverse directions, at a predefined scan rate, while monitoring the current. One full cycle, a partial cycle, or a series of cycles can be performed. While platinum is the preferred electrode material, other electrodes, such as gold, silver, iridium, or graphite, could be used. Although, cyclic voltammetric techniques are used, other pulse techniques such as differential pulse voltammetry or square wave voltammetry may increase the speed and sensitivity of measurements.

Embodiments of the present specification expressly cover any and all forms of automatically sampling and measuring, detecting, and analyzing an output fluid, or the headspace above the output fluid. For example, such automated detection can be achieved by integrating a mini-gas chromatography (GC) measuring device that automatically samples air in the output container, transmits it to a GC device optimized for the specific solvents used in the delipidation process, and, using known GC techniques, analyzes the sample for the presence of the solvents.

Referring back to FIG. 2, suitable materials for use in any of the apparatus components as described herein include materials that are biocompatible, approved for medical applications that involve contact with internal body fluids, and in compliance with U.S. PVI or ISO 10993 standards. Further, the materials do not substantially degrade from, for instance, exposure to the solvents used in the present specification, during at least a single use. The materials are sterilizable either by radiation or ethylene oxide (EtO) sterilization. Such suitable materials are capable of being formed into objects using conventional processes, such as, but not limited to, extrusion, injection molding and others. Materials meeting these requirements include, but are not limited to, nylon, polypropylene, polycarbonate, acrylic, polysulfone, polyvinylidene fluoride (PVDF), fluoroelastomers such as VITON, available from DuPont Dow Elastomers L.L.C., thermoplastic elastomers such as SANTOPRENE, available from Monsanto, polyurethane, polyvinyl chloride (PVC), polytetrafluoroethylene (PTFE), polyphenylene ether (PFE), perfluoroalkoxy copolymer (PFA), which is available as TEFLON PFA from E.I. du Pont de Nemours and Company, and combinations thereof.

Valves 215, 215a, 215b, 215c, 215d, 215e, 215f, 215g, 216 and any other valve used in each embodiment may be composed of, but are not limited to, pinch, globe, ball, gate or other conventional valves. In some embodiments, the valves are occlusion valves such as Acro Associates' Model 955 valve. However, the present specification is not limited to a valve having a particular style. Further, the components of each system described in accordance with embodiments of the present specification may be physically coupled together or coupled together using conduits that may be composed of flexible or rigid pipe, tubing or other such devices known to those of ordinary skill in the art.

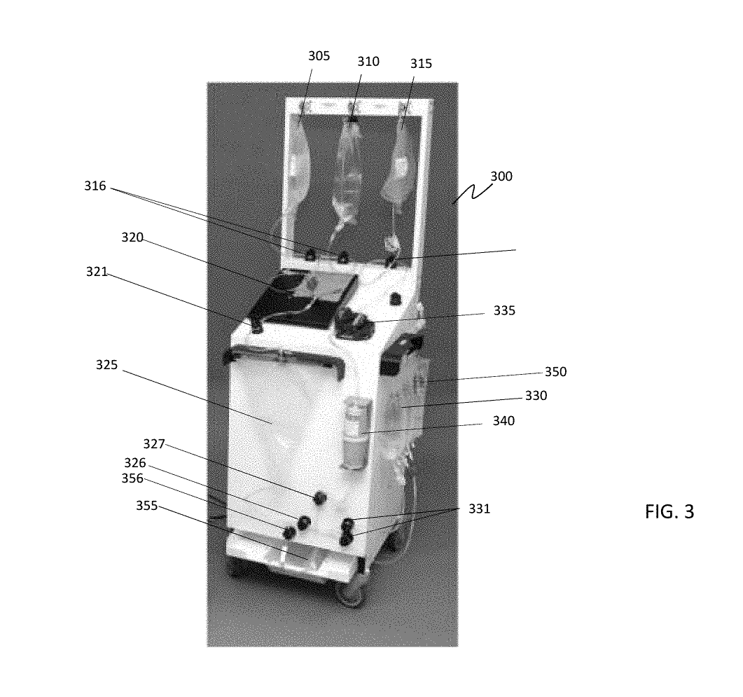

FIG. 3 illustrates an exemplary configuration of a system used in accordance with some embodiments of the present specification to achieve the processes disclosed herein. Referring to FIG. 3, a configuration of basic components of the HDL modification system 300 is shown. A fluid input 305 is provided and connected via tubing to a mixing device 320. A solvent input 310 is provided and also connected via tubing to a mixing device 320. Preferably valves 316 are used to control the flow of fluid from fluid input 305 and solvent from solvent input 310. It should be appreciated that the fluid input 305 preferably contains any fluid that includes HDL particles, including plasma having LDL particles or devoid of LDL particles, as discussed above. It should further be appreciated that solvent input 310 can include a single solvent, a mixture of solvents, or a plurality of different solvents that are mixed at the point of solvent input 310. While depicted as a single solvent container, solvent input 310 can comprise a plurality of separate solvent containers. The types of solvents that are used and preferred are discussed above.

The mixer 320 mixes fluid from fluid input 305 and solvent from solvent input 310 to yield a fluid-solvent mixture. Preferably, mixer 320 is capable of using a shaker bag mixing method with the input fluid and input solvent in a plurality of batches, such as 1, 2, 3 or more batches. Once formed, the fluid-solvent mixture is directed, through tubing and controlled by at least one valve 321, to a separator 325. In a preferred embodiment, separator 325 is capable of performing bulk solvent separation through gravity separation in a funnel-shaped bag.

In the separator 325, the fluid-solvent mixture separates into a first layer and second layer. The first layer comprises a mixture of solvent and lipid that has been removed from the HDL particles. The second layer comprises a mixture of residual solvent, modified HDL particles, and other elements of the input fluid. One of ordinary skill in the art would appreciate that the composition of the first layer and the second layer would differ based upon the nature of the input fluid. Once the first and second layers separate in separator 325, the second layer is transported through tubing to a solvent extraction device 340. Preferably, a pressure sensor 326 and valve 327 is positioned in the flow stream to control the flow of the second layer to the solvent extraction device 340.

Preferably, a glucose input 330 and saline input 350 is in fluid communication with the fluid path leading from the separator 325 to the solvent extraction device 340. A plurality of valves 331 is also preferably incorporated in the flow stream from the glucose input 330 and saline input 350 to the tubing providing the flow path from the separator 325 to the solvent extraction device 340. Glucose and saline are incorporated into the present specification in order to prime the solvent extraction device 340 prior to operation of the system. Where such priming is not required, the glucose and saline inputs are not required. Also, one of ordinary skill in the art would appreciate that the glucose and saline inputs can be replaced with other primers if the solvent extraction device 340 requires it.

The solvent extraction device 340 is preferably a charcoal column designed to remove the specific solvent used in the solvent input 310. An exemplary solvent extraction device 340 is an Asahi Hemosorber charcoal column. A pump 335 is used to move the second layer from the separator 325, through the solvent extraction device 340, and to an output container 315. The pump is preferably a peristaltic pump, such as a Masterflex Model 77201-62.

The first layer is directed to a waste container 355 that is in fluid communication with separator 325 through tubing and at least one valve 356. Additionally, other waste, if generated, can be directed from the fluid path connecting solvent extraction device 340 and output container 315 to waste container 355.

Preferably, an embodiment of the present specification uses gravity, wherever practical, to move fluid through each of the plurality of components. For example, preferably gravity is used to drain the input plasma 305 and input solvent 310 into the mixer 320. Where the mixer 320 comprises a shaker bag and separator 325 comprises a funnel bag, fluid is moved from the shaker bag to the funnel bag and, subsequently, to the waste container 355, if appropriate, using gravity.

In general, the present specification preferably comprises configurations wherein all inputs, such as input plasma and input solvents, disposable elements, such as mixing bags, separator bags, waste bags, solvent extraction devices, and solvent detection devices, and output containers are in easily accessible positions and can be readily removed and replaced by a technician.

To enable the operation of the above described embodiments of the present specification, it is preferable to supply a user of such embodiments with a packaged set of components, in kit form, comprising each component required to practice embodiments of the present specification. The kit may include an input fluid container (i.e. a high density lipoprotein source container), a lipid removing agent source container (i.e. a solvent container), disposable components of a mixer, such as a bag or other container, disposable components of a separator, such as a bag or other container, disposable components of a solvent extraction device (i.e. a charcoal column), an output container, disposable components of a waste container, such as a bag or other container, solvent detection devices, and, a plurality of tubing and a plurality of valves for controlling the flow of input fluid (high density lipoprotein) from the input container and lipid removing agent (solvent) from the solvent container to the mixer, for controlling the flow of the mixture of lipid removing agent, lipid, and particle derivative to the separator, for controlling the flow of lipid and lipid removing agent to a waste container, for controlling the flow of residual lipid removing agent, residual lipid, and particle derivative to the extraction device, and for controlling the flow of particle derivative to the output container.

In one embodiment, a kit comprises a plastic container having disposable components of a mixer, such as a bag or other container, disposable components of a separator, such as a bag or other container, disposable components of a waste container, such as a bag or other container, and, a plurality of tubing and a plurality of valves for controlling the flow of input fluid (high density lipoprotein) from the input container and lipid removing agent (solvent) from the solvent container to the mixer, for controlling the flow of the mixture of lipid removing agent, lipid, and particle derivative to the separator, for controlling the flow of lipid and lipid removing agent to a waste container, for controlling the flow of residual lipid removing agent, residual lipid, and particle derivative to the extraction device, and for controlling the flow of particle derivative to the output container. Disposable components of a solvent extraction device (i.e. a charcoal column), the input fluid, the input solvent, and solvent extraction devices may be provided separately.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A method for delaying a progression of, stabilizing, or improving symptoms related to Alzheimer's Disease (AD) in a patient, comprising:
monitoring a pathophysiological change indicative of AD in a patient;
based on said monitoring, determining if amyloid plaque is present in a perivascular space of the patient;
determining an extent of amyloid plaque in said perivascular space; and,
based on the presence of amyloid plaque in the perivascular space of the patient, determining a treatment protocol for the patient, wherein the treatment protocol comprises administering to the patient a high density lipoprotein composition derived from mixing a blood fraction with a lipid removing agent.

2. The method of claim 1, wherein diagnostic imaging is used to determine the presence and extent of amyloid plaque in the perivascular space of the patient.

3. The method of claim 1, wherein the high density lipoprotein composition is derived by
obtaining the blood fraction from the patient, wherein the blood fraction has high-density lipoproteins;
mixing the blood fraction with the lipid removing agent to yield modified high-density lipoproteins;
separating the modified high-density lipoproteins; and
delivering the modified high-density lipoproteins to the patient.

4. The method of claim 1, further comprising:
connecting the patient to a device for withdrawing blood;
withdrawing blood from the patient; and
separating blood cells from the blood to yield the blood fraction containing high density lipoproteins and low density lipoproteins.

5. The method of claim 3, wherein the modified high density lipoproteins have an increased concentration of pre-beta high density lipoproteins relative to the high density lipoproteins from the blood fraction prior to mixing.

6. The method of claim 3, wherein the modified high density lipoproteins have a concentration of alpha high density lipoproteins in addition to pre-beta high density lipoproteins from the blood fraction prior to mixing.

7. The method of claim 1 wherein the pathophysiological change is indicated by an accumulation of plaque in the perivascular space of the patient resulting in cerebral amyloid angiopathy.

8. The method of claim 1 wherein the high density lipoprotein composition derived from mixing the blood fraction with the lipid removing agent is delivered to the patient via infusion therapy in a dosage ranging from 1 mg/kg to 250 mg/kg.

9. The method of claim 1 wherein the high density lipoprotein composition derived from mixing the blood fraction of the patient with the lipid removing agent is delivered to the patient via infusion therapy at a rate of 999 mL/hour+/−100 mL/hr.

10. The method of claim 1 further comprising determining a severity of AD in the patient using at least one of global functioning, cognitive functioning, activities of daily living, or behavioral assessments.

11. The method of claim 1, wherein after administering to the patient the high density lipoprotein composition, the patient experiences a decrease in the accumulation of amyloid plaque in the perivascular space.

12. The method of claim 1 wherein after administering to the patient the high density lipoprotein composition, a rate of degeneration of the patient's physiological and/or cognitive parameters indicative of AD stabilizes and does not experience a further decrease.

13. The method of claim 1 wherein after administering to the patient the high density lipoprotein composition, a rate of degeneration of the patient's physiological and/or cognitive parameters indicative of AD, slows down relative to a rate of degeneration of the patient's physiological and/or cognitive parameters indicative of AD before administering to the patient the high density lipoprotein composition.

14. The method of claim 1, wherein after administering to the patient the high density lipoprotein composition, the patient's physiological and/or cognitive symptoms indicative of AD improve relative to the patient's physiological and/or cognitive symptoms indicative of AD before administering to the patient the high density lipoprotein composition.

15. The method of claim 1, wherein the high density lipoprotein composition is derived by obtaining the blood fraction from an individual other than the patient, wherein the blood fraction has high-density lipoproteins;
mixing the blood fraction with the lipid removing agent to yield modified high-density lipoproteins;
separating the modified high-density lipoproteins; and
delivering the modified high-density lipoproteins to the patient.

16. A method for delaying the progression of, stabilizing, or improving symptoms related to Alzheimer's Disease (AD) in a patient, comprising:
monitoring a pathophysiological change indicative of AD, or a potential future onset of AD, in the patient;
based on said monitoring, determining if amyloid plaque is present in a perivascular space of the patient;
based on the determination of the presence of amyloid plaque in the perivascular space of the patient, determining a treatment protocol for the patient, wherein the treatment protocol comprises administering to the patient a high density lipoprotein composition derived from mixing a blood fraction, having unmodified high density lipoproteins, with a lipid removing agent to yield modified high density lipoproteins, wherein the modified high density lipoproteins have an increased concentration of pre-beta high density lipoprotein relative to the unmodified high density lipoproteins.

17. The method of claim 16, wherein the composition is derived by
obtaining the blood fraction from the patient;
mixing the blood fraction with the lipid removing agent to yield the modified high-density lipoproteins;
separating the modified high-density lipoproteins; and
delivering the modified high-density lipoproteins to the patient.

18. The method of claim 16, further comprising:
connecting the patient to a device for withdrawing blood;
withdrawing blood from the patient; and
separating blood cells from the blood to yield the blood fraction containing low density lipoproteins and the high density lipoproteins.

19. The method of claim 16, wherein the composition is derived by
obtaining the blood fraction from an individual other than the patient;
mixing the blood fraction with the lipid removing agent to yield the modified high-density lipoproteins;
separating the modified high-density lipoproteins; and
delivering the modified high-density lipoproteins to the patient.

20. A method for improving an impairment of cognitive function indicative of Alzheimer's Disease (AD) in a patient, comprising:
determining if amyloid plaque is present in a perivascular space of the patient;
determining an extent or severity of cognitive impairment in the patient using at least one of a global, cognitive, functional or behavioral assessment test; and,
based on the determination of the presence of amyloid plaque in the perivascular space of the patient and said extent or severity of cognitive impairment in the patient, determining a treatment protocol for the patient, wherein the treatment protocol comprises administering to the patient a high density lipoprotein composition derived from mixing a blood fraction of the patient with a lipid removing agent.
